(19) United States
(12) Patent Application Publication
(10) Pub. No.: US 2016/0068598 A1
(43) Pub. Date: Mar. 10, 2016

(54) ANTI-CD19 COMPOSITIONS AND METHODS FOR TREATING CANCER

(71) Applicant: University of Louisville Research Foundation, Inc., Louisville, KY (US)

(72) Inventors: Jun Yan, Louisville, KY (US); Chuanlin Ding, Louisville, KY (US)

(21) Appl. No.: 14/946,253

(22) Filed: Nov. 19, 2015

Related U.S. Application Data
(63) Continuation of application No. 14/251,263, filed on Apr. 11, 2014.
(60) Provisional application No. 61/811,524, filed on Apr. 12, 2013.

(51) Int. Cl. C07K 16/28; C12N 9/12

(52) U.S. Cl. CPC C07K 16/2803; C12N 9/12; C07K 2317/622; C07K 2319/30; C07K 2319/33; C12Y 207/10001

(57) ABSTRACT

A composition is provided that includes an anti-CD19 single chain variable fragment (scFv) conjugated to a tumor-associated antigen. The antigen can comprise a Her-2/neu protein, such as a Her-2/neu extracellular domain. Methods of eliciting an immune response are also provided along with methods of treating, or limiting the occurrence of, cancer in a subject.

### RELATED APPLICATION

This application is a continuation application of U.S. application Ser. No. 14/251,263, which claims priority from U.S. Provisional Application Ser. No. 61/811,524, filed Apr. 12, 2013, the entire disclosure of which is incorporated by this reference.

### GOVERNMENT INTEREST

This invention was made with government support under grant no. NCI R01CA86412 awarded by the National Institutes of Health. The government has certain rights in the invention.

### TECHNICAL FIELD

The presently-disclosed subject matter relates to anti-CD19 compositions and methods for treating a cancer. In particular, the presently-disclosed subject matter relates to anti-CD19 compositions and methods for treating a cancer that make use of an anti-CD19 single chain variable fragment (scFv) conjugated to a tumor-associated antigen.

### BACKGROUND

Activation of the immune system is thought to be a target for cancer therapy, but developing effective cancer vaccines has proven to be a daunting task. Yet, the recent approval of the first therapeutic cancer vaccine by the U.S. Food and Drug Administration (FDA), Sipuleucel-T, a vaccine for the treatment of asymptomatic metastatic castrate-resistant advanced prostate cancer with a modest clinical benefit in some patients, has re-energized research into more potent cancer vaccine development. Indeed, many cancer vaccine platforms are currently being evaluated in pre-clinical animal models or in clinical trials, including protein or peptide-pulsed dendritic cell (DC)-based vaccines. The DC-based vaccine platform normally requires leukapheresis and further in vitro expansion of DCs. Drawbacks of this approach include large-scale preparation of clinical grade DCs, the choice of DC subsets, and DC-related trafficking. In contrast, anti-tumor mAb therapy has achieved clinical promise and now is widely used in oncology patient care. Thus, it would be desirable if tumor vaccines could elicit long-lasting anti-tumor humoral responses as well as T cell responses.

B cells are capable of eliciting anti-tumor responses by the production of antibodies (Abs) as well as serving as APCs to induce CD4 T cell responses. In addition, B cells can present antigen (Ag) to cross-prime CD8 T cells for expansion and activation. Ag activation of B cells has been shown to enhance the expression of co-stimulatory molecules, principally CD86, which is essential for B cells' ability to break T cell tolerance. However, the role of B cells in tumor development has been controversial. Certain studies have shown that therapeutic depletion of B cells enhances B16 melanoma growth in mice. In contrast, however, in a skin squamous carcinoma model, B cells, predominantly the Abs produced by B cells, promote tumor progression via triggering chronic inflammation. Nevertheless, the ability of B cells to induce both Ab production and T cell responses makes B cells a desirable cell subset for cancer vaccine development.

To date, however, conventional Ab therapy has proven to be costly and patients frequently develop acquired resistance over time. Further, in some cases of Ab therapy resistance, T cell responses have been shown to be essential. Thus, vaccination that generates a sustained Ab response as well as T cell response may be more useful and economical in treating, or limiting their occurrence of, diseases such as cancer.

### SUMMARY

The presently-disclosed subject matter meets some or all of the above-identified needs, as will be apparent to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter relates to treating and limiting the occurrence of cancer or tumor cells in a subject based on targeting B cells. In various different embodiments, the presently-disclosed subject matter includes a composition which targets B cells to induce antibody (Ab) production and T cell responses, which, in turn, are capable of targeting tumor cells. In other embodiments, the presently-disclosed subject matter relates to a unique composition which comprises an anti-CD19 single chain variable fragment (scFv) conjugated to a tumor-associated antigen. The associated antigen can comprise a Her-2/neu protein such as, in some embodiments, a Her-2/neu extracellular domain.

In some embodiments of the presently-disclosed subject matter, a pharmaceutical composition is provided that includes an anti-CD19 single chain variable fragment (scFv) conjugated to a tumor-associated antigen, and a pharmaceutically-acceptable vehicle, carrier, or excipient. The composition may be administered to a subject to treat or limit the occurrence of cancer or tumor cells in a subject. In some embodiments, the composition can be provided in the form of a vaccine.

Further provided, in some embodiments of the presently-disclosed subject matter, are methods of eliciting an immune response. In some embodiments, a method of eliciting an immune response is provided that comprises contacting a B cell with an effective amount of a composition comprising an anti-CD19 single chain variable fragment (scFv) conjugated to a tumor-associated antigen to thereby elicit the immune response. In some embodiments, the tumor associated antigen comprises a Her-2/neu protein such as, in some embodiments, a Her-2/neu extracellular domain. In some embodiments of the methods of eliciting an immune response, the immune response includes B cell antibody generation, T cell activation, T cell differentiation, or combinations thereof. In some embodiments of the methods, the immune response includes an increase in an amount of one or more inflammatory cytokines.

Still further provided in some embodiments of the presently-disclosed subject matter are methods of treating a cancer or limiting the occurrence or progression of cancer in a subject. In some embodiments, a method of treating or limiting the occurrence or progression of cancer is provided that comprises administering to a subject in need thereof an effective amount of a composition comprising an anti-CD19 single chain variable fragment (scFv) conjugated to a tumor-associated antigen. In some embodiments, the tumor associated antigen administered as part of the composition comprises a Her-2/neu protein such as, in some embodiments, a Her-2/neu extracellular domain. In some embodiments, the cancer is a breast cancer.

Further features and advantages of the present invention will become evident to those of ordinary skill in the art after a study of the description, figures, and non-limiting examples in this document.

### BRIEF DESCRIPTION OF THE DRAWINGS

FIGS. 1A-1E include schematic diagrams and graphs showing that CD19 scFv miniAb specially binds to B cells with high binding affinity. FIG. 1A is a schematic view of fusion protein constructs. FIG. 1B and FIG. 1C are blots showing lysates of BL21 (DE3)Plyss transfected with expression plasmids for CD19-scFv, her-2/neu P3-4, CD19-scFv-p3-4 (FIG. 1B), OVA and CD19-scFv-OVA (FIG. 1C), in which the lysates were purified and then western blotted using Abs against His-Tag, her-2/neu or OVA. FIG. 1D includes a series of graphs showing in vitro B cell binding in which splenocytes were incubated with scFv, P3-4, scFv-p3-4, biotin-OVA, biotin-scFv-OVA followed by APC-anti-mouse B220 and Oregon-green-Herceptin or PE-streptavidin, where cells were washed and assessed by flow cytometer and for in vivo binding, biotin-labeled-proteins were i.v. injected into mice, peripheral blood drawn at 10 min after injection, cells stained with APC-anti-mouse B220 and PE-streptavidin and assessed by flow cytometer. FIG. 1E includes graphs showing determination of protein K_{D} values by Line weaver-Burk analysis.

FIGS. 2A-2C include images and graphs showing that antigen bound to B cells via CD19 is co-localized with the BCR and that co-engagement of the BCR and CD19 activates B cells. FIG. 2A shows purified B cells that were cytospin to slides incubated with scFv-p3-4 fusion protein for 30 min and stained with biotinylated anti-her2/neu followed by Alexa Fluor 594-streptavidin (red) and Alexa Fluor 488-anti-IgM (green). FIG. 2B includes a series of flow cytometry graphs of B cells stimulated with fusion proteins as indicated for 24 h, in which surface markers were assessed by flow cytometry (data shown are one from 3 independent experiments). FIG. 2C includes a series of graphs from three representative experiments from supernatants from B cells stimulated with fusion proteins scFv, p3-4, scFv-p3-4, or medium alone for 48 h were harvested and assayed for cytokines IL-6, IL-12p40, and TNF-α by ELISA (error bars represent s.e.m.).

FIGS. 3A-3D include graphs showing T-cell responses mediated by scFv-OVA, where FIGS. 3A and 3B correspond to splenocytes from OT-I or OT-II mice labeled with CFSE and then stimulated with different concentrations of OVA or scFv-OVA for 3 days and the turnover of T cells and intracellular IFN-γ staining from splenic cells examined by flow cytometer, (cells gated on CD8⁺ or CD4⁺ populations), FIG. 3A showing T-cell proliferation overlay histogram from medium, OVA or scFv-OVA (A), and FIG. 3B showing IFN-γ production of CD4⁺ and CD8⁺. FIG. 3C corresponds to 2×10⁶ CFSE-labeled naive CD4 OT-II or CD8 OT-I T cells i.v. adoptively transferred into naive C57Bl/6 mice (n=3), followed by, the next day, mice injected with a single dose of OVA or scFv-OVA (for OT-I T cells, 5 μg protein/mouse; for OT-II T cells, 20 μg protein/mouse) or PBS, and recipient mice killed after 3 days and turnover of T cells from splenic cells examined by flow cytometer, wherein cells were gated on CFSE-positive population. T-cell proliferation shown in overlay histogram was from PBS, OVA, or scFv-OVA (shown from three representative experiments).

FIGS. 4A-4E includes graphs showing that scFv-p3-4 fusion protein elicits enhanced her-2/neu-specific humoral responses. FIG. 4A corresponds to mice (n=3) immunized with scFv, P3-4, scFv-p3-4 (50 μg/mouse) for 3 times at a 1-week interval, mice were bled at day 7 (IgM) and day 21 (IgG) and sera measured for her2/neu-specific Abs by ELISA. FIG. 4B corresponds to SKOV-3 tumor cells stained with sera (1:20) from pre- and post-scFv, P3-4 or scFv-p3-4-immunized mice followed with secondary anti-mouse IgG-FITC and examined by flow cytometer. (Summarized mean fluorescent intensity (MFI) shown in bar graph on the right). FIG. 4C corresponds to SKOV3 tumor cells incubated with heat inactivated post-immune serum followed by SCID mice serum, fITC-anti-mouse C3 antibody was added; and C3 deposition was measured by flow cytometry summarized MFI is shown in the bar graph to the right. FIG. 4D is a graph from Ab competitive inhibition assay showing that sera from scFv-p3-4-immunized mice are capable of inhibiting Herceptin-mediated binding. (percent inhibition is shown). FIG. 4E is a plot showing tumor inhibition using 1×10⁴ SKBR-3 cells placed into the wells of the Acea 16-well plates for 24 h, ten μl of heat-inactivated fusion protein immunized serum or pre-immune serum were added to cells and incubated for 170 h, in which inhibition of tumor cell growth was calculated by measuring the relative decrease in current impedance among wells containing immune serum and wells containing pre-immune serum (representative of 3 experiments).

FIGS. 5A-5D include graphs showing increased IFN-γ-producing CD8 T cells, IL-4-producing CD4 T cells, T cell proliferation, and augmented in vivo cytolytic activity elicited by targeting of her-2/neu to B cells. FIGS. 5A and 5C correspond to BALB/c mice (n=3) immunized with scFv, P3-4 or scFv-p3-4 for 4 times at a 1-week interval, in which splenocytes from immunized mice were stimulated with scFv-p3-4 (20 μg/ml) for 3 days, wherein FIG. 5A corresponds to intracellular IFN-γ of CD4 and CD8 (A) being carried out, FIG. 5B showing IL-4 production and FIG. 5C showing measurement of ³H-thymidine proliferation. Cells from immunized mice (n=3-5) as described above were stimulated with ConA, IL-2, and IL-4 and then re-stimulated with immobilized CD3 and soluble CD28 mAbs, followed by, Intracellular IL-4 staining FIG. 5D are graphs showing in vivo cytotoxicity, in which CFSE labeled syngeneic B cells pulsed with (CFSE^{high}) or without scFv-p3-4 (CFSE^{low}) injected intravenously into immunized mice; mice sacrificed 24 h after transfer, splenocytes harvested and assessed by flow cytometry; and cells gated on CFSE-positive cells (Representative histograms and summarized % of cytotoxicity were shown with data representative of 3 experiments).

FIGS. 6A-6H includes graphs showing reduced tumor burden and enhanced tumor-free survival upon scFv-p3-4 vaccination. FIGS. 6A and 6B correspond to BALB/c mice vaccinated with scFv, P3-4 or scFv-p3-4 for 4 times at a 1-week interval. PBS-immunized mice were used as control, where, at day 28, immunized mice were challenged s.c. with 1×10⁵ D2F2/E2 tumor cells wherein FIG. 6A is a graph showing tumor growth and FIG. 6B is a graph showing recorded survival. FIGS. 6C and 6D correspond to BALB/c mice challenged s.c. with 1×10⁵ D2F2/E2 tumor cells, in which mice were treated with scFv, P3-4 or scFv-p3-4 for 4 times at a 1-week interval when the tumor size (diameter) reached 2-3 mm, wherein FIG. 6C is a graph showing tumor growth and FIG. 6D is a graph showing recorded survival. FIGS. 6E and 6F correspond to BALB/c mice first injected intraperitoneally with anti-CD4 mAb (250 μg), anti-CD8 (500 μg) or isotype control mAb (250 μg) on day −3, in which mice were then immunized with different fusion proteins, depletion mAbs or isotype control mAb was further injected 3 days before boost immunization, and after 4 times immunization, mice were challenged s.c. with 1×10⁵ D2F2/E2 tumor cells, wherein FIG. 6E is a graph showing tumor growth and FIG. 6F is a graph showing recorded survival. FIGS. 6G and 6H correspond to C57Bl/6 mice challenged s.c. with 5×10⁵ EO771/E2 tumor cells, in which tumor-bearing mice were treated with scFv, P3-4 or scFv-p3-4 for 4 times at a 1-week interval when the tumor size (diameter) reached 2-3 mm, wherein FIG. 6G is a graph showing tumor growth and FIG. 6H is a graph showing recorded survival.

FIGS. 7A-7F include images and graphs showing that antibodies elicited by targeting her-2/neu different ECD domains via CD19 scFV have differential anti-tumor effect in vitro. FIG. 7A are blots from purified fusion proteins scFv-D1, scFv-D2, scFv-D3 and scFv-D4 blotted with his tag mAb or her-2/neu Ab, FIG. 7B are graphs for binding studies for in vitro B cell binding, splenocytes incubated with biotinylated scFv-D1, scFv-D2, scFv-D3, and scFv-D4 followed by APC-anti-mouse B220 and FITC-streptavidin, and FIG. 7C are graphs showing immune response from mice immunized with scFv-D1, scFv-D2, scFv-D3 and scFv-D4 (50 μg/mouse) for 4 times at 1-week intervals, in which sera were collected at day 28 and then measured for her2/neu-specific Abs by ELISA. FIG. 7D is a graph showing tumor growth inhibition from a study including 1×10⁴ SKBR-3 cells placed into the wells of the Acea 16-well plates for 24 h, heat-inactivated immune serum (1:20), pre-immune serum (1:20), or Herceptin (10 μg/ml) added to wells and incubated for indicated times, wherein inhibition of tumor cell growth was calculated by measuring the relative decrease in current impedance among wells containing post-immune serum and wells containing pre-immune serum. FIG. 7E is a graph from data collected by immunoblotting of phosphorylated Akt (p-Akt) in post-immune serum-treated (1:10 dilution), Herceptin (2 μg/ml) or medium-treated SKBR-3 cells. β-Actin served as loading control and Densitometric quantification is also shown (n=4). (*p<0.05, **p<0.01, ***p<0.001. n.s. not significant). FIG. 7F is a graph showing data collected from study of FIG. 7E.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is based, at least in part, on the discovery that a composition comprising an anti-CD19 single chain variable fragment (scFv) conjugated to a tumor-associated antigen is capable of providing unexpected benefits in terms of its ability to generate an increased and/or more advantageous immune response as compared with prior methods that make use of whole CD19 antibodies to target B cells. As would be recognized by those skilled in the art, CD19 is a B cell-specific protein that is expressed at almost every stage of B cell development. Further, CD19 on the B cell surface is important for B cell antigen presentation. It has now been determined, however, that conjugating an anti-CD19 scFv to specific tumor-associated antigens leads to a more efficient antigen presentation by B cells and a more potent CD4 and CD8 T cell activation.

The presently-disclosed subject matter thus includes compositions comprising an anti-CD19 single chain variable fragment (scFv) conjugated to a tumor-associated antigen. In some embodiments of the compositions disclosed herein, the tumor-associated antigen comprises a Her-2/neu protein. In some embodiments, the Her-2/neu protein comprises a Her-2/neu extracellular domain, such as a Herceptin-binding domain that includes amino acids 241-386 of the her2/neu protein. Of course, any number of tumor-associated antigens may be used in accordance with the presently-disclosed subject matter and attached to an anti-CD19 scFv. For example, in further embodiments, the tumor associated antigen conjugated to the anti-CD19 scFv is the tumor-associated antigen MUC1 (mucin 1) or the tumor-associated antigen EGFR (epidermal growth factor receptor). See, e.g., Ding C., et al. Blood. 2008 Oct. 1; 112(7): 2817-25, which is incorporated herein by reference in its entirety.

One aspect of the presently-disclosed subject matter thus pertains, in some embodiments, to fusion proteins and nucleic acids (e.g., DNA) encoding the fusion proteins. The term “fusion protein” is intended to describe at least two polypeptides, typically from different sources, which are operatively linked. With regard to the polypeptides of the compositions, the term “conjugated” is used herein interchangeably with the term “operatively linked” to refer to two polypeptides that are connected in a manner such that each polypeptide can serve its intended function. Typically, the two polypeptides are covalently attached through peptide bonds and can be produced by standard recombinant or chemical synthesis techniques. For example, using recombinant techniques, a DNA molecule encoding a first polypeptide can be ligated to another DNA molecule encoding the second polypeptide, and the resultant hybrid DNA molecule can be expressed in a host cell to produce the fusion protein. The DNA molecules are generally ligated to each other in a 5′ to 3′ orientation such that, after ligation, the translational frame of the encoded polypeptides is not altered (i.e., the DNA molecules are ligated to each other in-frame).

In some embodiments of the presently-disclosed subject matter, pharmaceutical compositions including the anti-CD19 scFV compositions described herein are further provided. In some embodiments, a pharmaceutical composition is provided that comprises an anti-CD19 scFV composition disclosed herein and a pharmaceutically-acceptable vehicle, carrier, or excipient. In some embodiments, the pharmaceutical composition is pharmaceutically-acceptable in humans. Also, as described further below, in some embodiments, the pharmaceutical composition can be formulated as a therapeutic composition for delivery to a subject.

A pharmaceutical composition as described herein preferably comprises a composition that includes a pharmaceutical carrier such as aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The pharmaceutical compositions used can take such forms as suspensions, solutions or emulsions in oily or aqueous vehicles, and can contain formulatory agents such as suspending, stabilizing and/or dispersing agents. Additionally, the formulations can be presented in unit-dose or multi-dose containers, for example sealed ampoules and vials, and can be stored in a frozen or freeze-dried or room temperature (lyophilized) condition requiring only the addition of sterile liquid carrier immediately prior to use.

In some embodiments, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, but are not limited to, microcrystalline cellulose, corn starch, sodium starch glycolate, and alginic acid. Tablet binders that can be used include acacia, methylcellulose, sodium carboxymethylcellulose, polyvinylpyrrolidone, hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation. Numerous techniques for formulating sustained release preparations are known to those of ordinary skill in the art and can be used in accordance with the present invention, including the techniques described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491, each of which is incorporated herein by this reference.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically-acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration, the compositions can take the form of capsules, tablets or lozenges formulated in conventional manner.

Various liquid and powder formulations can also be prepared by conventional methods for inhalation into the lungs of the subject to be treated or for intranasal administration into the nose and sinus cavities of a subject to be treated. For example, the compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. Capsules and cartridges of, for example, gelatin for use in an inhaler or insufflator may be formulated containing a powder mix of the desired compound and a suitable powder base such as lactose or starch.

The compositions can also be formulated as a preparation for implantation or injection. Thus, for example, the compositions can be formulated with suitable polymeric or hydrophobic materials (e.g., as an emulsion in an acceptable oil) or ion exchange resins, or as sparingly soluble derivatives (e.g., as a sparingly soluble salt).

The compositions can further be formulated as topical semi-sold ointment or cream formulations can contain a concentration of the presently-described compositions in a carrier such as a pharmaceutical cream base. Various formulations for topical use include drops, tinctures, lotions, creams, solutions, and ointments containing the active ingredient and various supports and vehicles. The optimal percentage of the therapeutic agent in each pharmaceutical formulation varies according to the formulation itself and the therapeutic effect desired in the specific pathologies and correlated therapeutic. In some embodiments, such ointment or cream formulations can be used for trans-dermal delivery of the pharmaceutical compositions described herein or for delivery to certain organs.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compositions can be administered by the drip method, whereby a formulation including a pharmaceutical composition of the presently-disclosed subject matter and a physiologically-acceptable excipient is infused. Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the composition can be prepared and administered as a suspension in an aqueous base or a pharmaceutically-acceptable oil base, such as an ester of a long chain fatty acid, (e.g., ethyl oleate).

In addition to the formulations described above, the anti-CD19 scFV compositions of the presently-disclosed subject matter can also be formulated as rectal compositions, such as suppositories or retention enemas, e.g., containing conventional suppository bases such as cocoa butter or other glycerides. Further, the anti-CD19 scFV compositions can also be formulated as a depot preparation by combining the compositions with suitable polymeric or hydrophobic materials (for example as an emulsion in an acceptable oil) or ion exchange resins, or as sparingly soluble derivatives, for example, as a sparingly soluble salt.

In some embodiments, the presently-disclosed compositions can be formulated as a vaccine using conventional vaccine preparation techniques which may include using suitable carriers and the like in the vaccine formulation. For example, injectable forms of the vaccine comprising the present compositions may include various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like.

Further provided, in some embodiments of the presently-disclosed subject matter, are methods for eliciting an immune response in a subject. As used herein, the term “immune response” includes responses which are caused, at least in part, or mediated, by the immune system of an individual. In some embodiments, a method for eliciting an immune response is provided that comprises administering to a subject in need thereof an effective amount of an anti-CD19 scFV composition of the presently-disclosed subject matter. For example, in some embodiments, the anti-CD19 scFV composition administered to a subject to elicit an immune response comprises an anti-CD19 scFV conjugated to a Her-2/neu protein, such as a Her-2/neu extracellular domain.

For administration of a therapeutic composition as disclosed herein (e.g., a composition comprising an anti-CD19 scFV conjugated to a Her-2/neu protein), conventional methods of extrapolating human dosage based on doses administered to a murine animal model can be carried out using the conversion factor for converting the mouse dosage to human dosage: Dose Human per kg=Dose Mouse per kg×12 (Freireich, et al., (1966) Cancer Chemother Rep. 50: 219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate kg factor. In an adult human, 100 mg/kg is equivalent to 100 mg/kg×37 kg/sq m=3700 mg/m2.

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, dermally (e.g., topical application), intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments of the therapeutic methods described herein, the therapeutic compositions are administered orally, intravenously, or intraperitoneally to thereby elicit an immune response and/or treat a disease or disorder, as described herein below.

Regardless of the route of administration, the compositions of the presently-disclosed subject matter typically not only include an effective amount of a therapeutic agent, but are typically administered in amount effective to achieve the desired response. As such, the term “effective amount” is used herein to refer to an amount of the therapeutic composition sufficient to produce a measurable biological response (e.g., an immune response). Actual dosage levels of active ingredients in a therapeutic composition of the presently-disclosed subject matter can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, N.J.; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

With further respect to the therapeutic methods described herein, including the above-described methods of eliciting an immune response, in some embodiments of the therapeutic methods, the immune response to the composition includes B cell antibody generation, T cell activation, T cell differentiation, and combinations thereof. In some embodiments, administering a composition comprising an anti-CD19 single chain variable fragment (scFv) conjugated to a tumor-associated antigen of the presently-disclosed subject matter increases an amount of an inflammatory cytokine in a subject. In some embodiments, the inflammatory cytokine can be interleukin-12 (IL-12), tumor necrosis factor-alpha (TNF-α), and/or interleukin-6 (IL-6).

Various methods known to those skilled in the art can be used to determine an increase in the amount of inflammatory cytokines in a subject. For example, in certain embodiments, the amounts of expression of an inflammatory cytokine in a subject can be determined by probing for mRNA of the gene encoding the inflammatory cytokine in a biological sample obtained from the subject (e.g., a tissue sample, a urine sample, a saliva sample, a blood sample, a serum sample, a plasma sample, or sub-fractions thereof) using any RNA identification assay known to those skilled in the art. Briefly, RNA can be extracted from the sample, amplified, converted to cDNA, labeled, and allowed to hybridize with probes of a known sequence, such as known RNA hybridization probes immobilized on a substrate, e.g., array, or microarray, or quantitated by real time PCR (e.g., quantitative real-time PCR, such as available from Bio-Rad Laboratories, Hercules, Calif.). Because the probes to which the nucleic acid molecules of the sample are bound are known, the molecules in the sample can be identified. In this regard, DNA probes for one or more of the mRNAs encoded by the inflammatory genes can be immobilized on a substrate and provided for use in practicing a method in accordance with the presently-disclosed subject matter.

With further regard to determining levels of inflammatory cytokines in samples, mass spectrometry and/or immunoassay devices and methods can also be used to measure the inflammatory cytokines in samples, although other methods can also be used and are well known to those skilled in the art. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety.

Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety. Mass spectrometry (MS) analysis can be used, either alone or in combination with other methods (e.g., immunoassays), to determine the presence and/or quantity of an inflammatory molecule in a subject. Exemplary MS analyses that can be used in accordance with the present invention include, but are not limited to: liquid chromatography-mass spectrometry (LC-MS); matrix-assisted laser desorption/ionization time-of-flight MS analysis (MALDI-TOF-MS), such as for example direct-spot MALDI-TOF or liquid chromatography MALDI-TOF mass spectrometry analysis; electrospray ionization MS (ESI-MS), such as for example liquid chromatography (LC) ESI-MS; and surface enhanced laser desorption/ionization time-of-flight mass spectrometry analysis (SELDI-TOF-MS). Each of these types of MS analysis can be accomplished using commercially-available spectrometers, such as, for example, triple quadropole mass spectrometers. Methods for utilizing MS analysis to detect the presence and quantity of peptides, such as inflammatory cytokines, in biological samples are known in the art. See, e.g., U.S. Pat. Nos. 6,925,389; 6,989,100; and 6,890,763 for further guidance, each of which are incorporated herein by this reference.

With still further regard to the various therapeutic methods described herein, although certain embodiments of the methods disclosed herein only call for a qualitative assessment (e.g., the presence or absence of the expression of an inflammatory cytokine in a subject), other embodiments of the methods call for a quantitative assessment (e.g., an amount of increase in the level of an inflammatory cytokine in a subject). Such quantitative assessments can be made, for example, using one of the above mentioned methods, as will be understood by those skilled in the art.

The skilled artisan will also understand that measuring an increase in the amount of a certain feature (e.g., cytokine levels) or an improvement in a certain feature (e.g., inflammation) in a subject is a statistical analysis. For example, an increase in an amount of inflammatory cytokines in a subject can be compared to control level of inflammatory cytokines, and an amount of inflammatory cytokines of greater than or equal to the control level can be indicative of an increase in the amount of inflammatory cytokines, as evidenced by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.]

Still further provided, in some embodiments, are methods for treating a cancer. In some embodiments, a method for treating a cancer is provided that comprises administering to a subject in need thereof an effective amount of an anti-CD19 scFV composition of the presently-disclosed subject matter (i.e., where the anti-CD19 scFV is conjugated to a tumor associated antigen). In some embodiments, the tumor associated antigen comprises a Her-2/neu protein, such as a Her-2/neu extracellular domain, such that the subject in need or treatment for breast cancer can be administered the composition to thereby elicit an immune response against the cancer. In some embodiments, administering the composition to the subject to treat the cancer comprises repeated administration to the subject.

As used herein, the terms “treatment” or “treating” relate to any treatment of a condition of interest (e.g., a cancer), including but not limited to prophylactic treatment and therapeutic treatment. As such, the terms “treatment” or “treating” include, but are not limited to: preventing a condition of interest or the development of a condition of interest; inhibiting the progression of a condition of interest; arresting or preventing the further development of a condition of interest; reducing the severity of a condition of interest; ameliorating or relieving symptoms associated with a condition of interest; and causing a regression of a condition of interest or one or more of the symptoms associated with a condition of interest.

As further non-limiting examples of the treatment of a cancer by a composition described herein, treating a cancer can include, but is not limited to, killing cancer cells, inhibiting the development of cancer cells, inducing apoptosis in cancer cells, reducing the growth rate of cancer cells, reducing the incidence or number of metastases, reducing tumor size, inhibiting tumor growth, reducing the available blood supply to a tumor or cancer cells, promoting an immune response against a tumor or cancer cells, reducing or inhibiting the initiation or progression of a cancer, increasing the lifespan of a subject with a cancer, or inhibiting or reducing the formation of DNA adducts by chemical carcinogens.

As used herein, the term “cancer” refers to all types of cancer or neoplasm or malignant tumors found in animals, including leukemias, carcinomas, melanoma, and sarcomas. By “leukemia” is meant broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. In some embodiments, the cancer is breast cancer.

In some embodiments, the compositions of the presently-disclosed subject matter can further be used in a method of limiting the occurrence of cancer in a subject. For example, in some embodiments, a method of limiting the occurrence of cancer in a subject is provided wherein an anti-CD19 scFv composition of the presently-disclosed subject matter is administered to a subject such that the tumor-associated antigen in the composition generates an immune response in the subject to thereby limit the occurrence of cancer in the subject. In other words, in some embodiments, the administration of a composition of the presently-disclosed subject matter can be used prophylactically to thereby limit the incidence or occurrence of cancer in a subject.

As used herein, the term “subject” includes both human and animal subjects. Thus, veterinary therapeutic uses are provided in accordance with the presently-disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples and experiments.

### EXAMPLES

### Materials and Methods

Mice and Cell Lines.

Balb/c, OVA TCR Tg OT-I, and OT-II Rag-deficient mice were purchased from the Jackson Laboratory (Bar Harbor, Me.). C57Bl/6 mice were purchased from the NCI (Frederick, Md.). All experimental mice were housed under specific pathogen-free conditions in the animal facility of University of Louisville and treated in accordance with the guidelines of the Institutional Animal Care and Use Committee (IACUC) of the University of Louisville.

Balb/c mammary carcinoma cell D2F2/E2 expressing human her-2/neu was kindly provided by Dr. Wei-Zen Wei (Karmanos Cancer Institute, Detroit, Mich.). Human her-2/neu expressing C57Bl/6 mammary tumor line EO771 (EO771/E2) was generated by stable transfection with human full-length her-2/neu cDNA plasmid (Rad Kevich-Brown (2009)). Human breast cancer cell SKBR-3 and human ovarian cancer cell SKOV3 from ATCC were maintained in DMEM supplemented with 10% heat-inactivated FCS, 2 mM L-glutamine, 100 U/ml penicillin, and 100 μg/ml streptomycin. Rat anti-mouse CD19 hybridoma (1D3) from ATCC was maintained in MEM supplemented with 10% FBS. BL21(DE3)Plyss competent cells were purchased from Novagen.

Generation of CD19 scFv miniAb and Fusion Proteins.

To generate CD19 scFv, total RNA was extracted from 1D3 and first strand cDNA was synthesized. VH and VL were amplified using primers in the following Table 1:

TABLE 1

| Name | Sequence | SEQ ID NO: |
|---|---|---|
| Vkappa | GAAGATCTCCACCATGGACATT | SEQ ID NO: 1 |
| start | CAGCTGACCCAGTCTCCA | |
| Jkappa | AGAGCCACCTCCGCCCCGTTTC | SEQ ID NO: 2 |
| with linker | AGTTCCAGCTTGGTGCC | |
| VH start | GGCGGAGGTGGCTCTARGTSMA | SEQ ID NO: 3 |
| with linker | RCTGVTWGSARCWGG | |
| VH stop | ACTAGTCGACTCATGAGGACAC | SEQ ID NO: 4 |
| | GGTGACCATGGTTCCTGGGCCCC | |

The single chain Fv (VL-VH) was then synthesized by overlapping PCR. PCR product was sequenced and further cloned into pET-20b (+) vectors in an NcoI site.

The full-length human c-ErbB-2 (Her-2/neu) cDNA was isolated from plasmid pCMV-ErbB-2 as a 4.4-kb EcoRI restriction fragment and was kindly provided by Dr. Wei. The pET-20b(+)-anti-CD19-scFv-c-ErbB-2 cDNA constructs that encode the herceptin-binding domain (from residues 475 to 652, designated as P3-4) were generated. Four different extracellular domains of ErbB-2 were also cloned into pET-20b (+) vectors using primers summarized in Table 2 below.

TABLE 2

| | Forward | Reverse |
|---|---|---|
| Her-2 ECD | AATGTCGACATGGAGCTGGCGG | AATCTCGAGACAGGGGTGGCAG |
| domain 1 | CCTTGTG (SEQ ID NO: 5) | GCCCGAGA (SEQ ID NO: 6) |
| Her-2 ECD | AATGTCGACTCTCCGATGTGTAA | AATCTCGAGTTGGTTGTGCAGGG |
| domain 2 | GGGCTCCC (SEQ ID NO: 7) | GGCAGAC (SEQ ID NO: 8) |
| Her-2 ECD | AATGTCGACGAGGTGACAGCAG | AATCTCGAGGTTCCGAAAGAGC |
| domain 3 | AGGATGGA (SEQ ID NO: 9) | TGGTCCCA (SEQ ID NO: 10) |
| Her-2 ECD | AATGTCGACCCGCACCAAGCTC | AATCTCGAGCGTCAGAGGGCTG |
| domain 4 | TGCTCCAC (SEQ ID NO: 11) | GCTCTCT (SEQ ID NO: 12) |
| OVA | AGGTCGACAGCATGTTGGTGCT | GACTCGAGAGGGGAAACACATC |
| | GTTGC (SEQ ID NO: 13) | TGCCA (SEQ ID NO: 14) |

The short OVA fragment containing OVA₂₅₇₋₂₆₄ and OVA₃₂₃₋₃₃₉ encoding sequence (residues 241-386) was amplified from plasmid pCMV-OVA and subcloned in-frame between the sal1 and xho1 restriction sites of pET-20b(+)-scFv using primers shown above.

pET-20b (+) constructs were transfected into BL21(DE3)Plyss cells and induced with 0.1 mM IPTG. The proteins were purified using His-Select Nickel Affinity Gel (Sigma) and LPS contamination was removed by Detoxi-Gel™ Endotoxin Removing Columns (Thermo Scientific). The proteins were dialyzed in PBS and analyzed with SDS-PAGE and Western Blot (WB). Endotoxin level was <0.5EU/mg as measured by LAL assay (Associations of Cape Cod, Inc).

Conjugation of Protein P3-4 to Anti-CD19 mAb.

Rat anti-mouse CD19 mAb (IgG2a) mAb was reduced in 20 mM dithiothreitol (DTT; Bio-Rad, Hercules, Calif.) at room temperature for 30 min and then separated from the reducing agent over a desalting column. LPS-free recombinant protein P3-4 was activated with succinimidyl 4-(N-maleimidomethyl)cyclohexane-1-carboxylate (SMCC; Pierce) according to the manufacturer's protocol and mixed with the reduced mAb for 1 hour at room temperature and then incubated overnight at 4° C. The unconjugated anti-CD19 mAb or free protein P3-4 was removed by size-exclusive column. The conjugates were examined for B-cell binding as assessed by flow cytometry.

Fusion Protein Binding Assay and Confocal Microscopy.

For in vitro binding assay, splenocytes were incubated with proteins scFv, P3-4, scFv-p3-4, OVA-biotin, scFv-OVA-biotin or biotin-labeled scFv-D1, scFv-D2, scFv-D3, scFv-D4 and then stained with oregon-green-labeled anti-Her2 Ab or PE-streptavidin and APC-anti-mouse B220. Cells were washed and assessed by flow cytometry. For the in vivo binding assay, mice were injected i.v. with biotinylated fusion proteins scFv, P3-4, scFv-p3-4, OVA or scFv-OVA. Peripheral blood was drawn at 10 min after injection. Cells were stained with APC-anti-mouse B220 and PE-streptavidin and assessed by flow cytometry. For determination of protein (scFv, scFv-p3-4, parental CD19 mAb) K_{D} values, B cells were incubated with different concentrations of proteins and then assayed by flow cytometry. Kd values were calculated by the following equation: 1/(F−F_{back})=1/Fₘₐₓ+(K_{D}/Fₘₐₓ)(1/[scFv], where F=fluorescence unit, F_{back}=background fluorescence and Fₘₐₓ was calculated from plot. For co-localization assays, B cells were incubated with scFv-p3-4 for 30 min and stained with biotinylated anti-her-2/neu followed by Alexa Fluor 594-streptavidin and Alexa Fluor 488-anti-IgM for 30 min at 4° C. Cells were analyzed on a Nikon confocal microscope.

In Vitro B Cell Culture.

Purified B cells were stimulated with scFv, P3-4, scFv-p3-4 or CD19 mAb conjugated P3-4 (1 μg/ml) for 24 h and then harvested to detect surface marker expression by flow cytometer. For cytokine assays, B cells were incubated for 48 h with different fusion proteins (20 μg/ml) and supernatants were harvested to measure cytokine levels by ELISA.

Detection of her-2/neu Abs by ELISA.

Ninety-six-well plates were coated with recombinant her-2/neu P1-4 protein (1 μg/well) overnight at 4° C. and blocked with 0.5% BSA/PBS. Pre- or post-immune sera from mice were diluted and further reacted with goat anti-mouse IgM or IgG horseradish peroxidase (HRP) conjugates (Southern Biotech, Birmingham, Ala.). The assays were subsequently developed by the addition of ABTS 1 Component Microwell Substrate (BioFX Laboratories, Owings Mills, Md.) and the OD450 was determined. To measure IgG isotype Abs, sera were diluted at 1:100 and further reacted with HRP-conjugated goat anti-mouse IgG1, IgG2a and IgG2b (Southern Biotech, Birmingham, Ala.). Ab concentrations were determined by generating a standard curve using serial dilutions of Herceptin.

Ab Competitive Inhibition Assay.

Micro-titer plates were coated with 1 μg/well recombinant her-2/neu P3-4 for overnight and then blocked with 0.5% BSA/PBS for 1 h at RT. Serially diluted immune sera (1:20, 1:40, 1:80) were added into wells for 1 h at RT and followed by biotin-Herceptin. Pre-immune sera were used as controls. The wells were incubated with streptavidin-HRP and ABTS 1 Component Microwell Substrate. OD450 was measured. The percent inhibition was calculated using formula: (ODₚᵣₑ−ODₚₒₛₜ)/ODₚᵣₑ×100%.

Complement Activation Assay.

1×10⁶ SKOV3 tumor cells were suspended in 100 μl HBSS with calcium and magnesium and incubated with inactivated-post/pre-immune sera (1:10 dilution) for 30 min at 37° C. SCID mouse sera as a source of complement were added and followed by FITC-anti-mouse C3 (MP Biomedical, Inc.) and measured by flow cytometry.

In Vitro Tumor Cell Growth Inhibition Assay and Western Blot (WB).

1×10⁴ SKBR-3 cells were placed into the wells of the Acea 16-well plates for 24 h. Ten μl of heat-inactivated pre- or post-immune sera were added to wells and incubated for indicated times. Herceptin was used as positive control. The inhibition of tumor cell growth was calculated by measuring the relative decrease in current impedance among wells containing post-immune serum and wells containing pre-immune serum only as described previously in Li (1997) and Solly (2004). The percent of inhibition was calculated using formula: (Cell indexₚᵣₑ−Cell indexₚₒₛₜ)/Cell indexₚᵣₑ×100%. For WB, SKBR-3 cells were treated with medium, heat-inactivated immune serum (1:10) or Herceptin (2 μg/ml) for 3 h at 37° C. Cells were lysed and immunoblot was performed with rabbit anti-Phospho-Akt Ab (Cell Signaling Technology Inc. Danvers, Mass.) or mouse anti-β-actin Ab (Sigma, St. Louis, Mo.). The blots were visualized using the ECL prime WB detection reagents (GE Healthcare Biosciences, Pittsburgh, Pa.).

T Cell Proliferation Assay.

For in vitro proliferation assays, splenocytes from OT-I or OT-II Tg mice were labeled with 10 μM CFSE (Molecular Probes, Carlsbad, Calif.) and then stimulated with varying concentrations of fusion proteins OVA or scFv-OVA. Three days later, turnover of T cells was examined by flow cytometry. For ³H-thymidine incorporation assays, splenocytes from immunized mice were cultured in 96-well plates (5×10⁵ cells/well) and stimulated with scFv-p3-4 (20 μg/ml) for 72 h. ³H-thymidine was added 16 h before 3 days culture and proliferation was measured by Scintillation counter (Packard). Stimulation index (SI) was used to show fold increase. SI was calculated using formula: cpmₑₓₚ/cpm_{control}. For in vivo proliferation assays, CD8 or CD4 T cells purified from OT-I or OT-II Tg mice were labeled with 10 μM CFSE. T cells (2×10⁶/mouse) were then adoptively transferred into recipient mice. OVA or scFv-OVA fusion proteins were injected into mice i.v. 24 h after adoptive transfer. Recipient mice were sacrificed after 3 days and the turnover of T cells was examined by flow cytometry.

Intracellular Cytokine Staining.

Intracellular cytokine staining was performed using BD Cytofix cytoperm kit with BD Golgiplug (BD Pharmingen, San Diego, Calif.) according to the manufacturer's protocol. For IFN-γ staining, cells were stimulated with OVA, scFv-OVA or scFv-p3-4 for 3 days, and then re-stimulated with PMA plus ionomycin for 4 h in the presence of Golgiplug and then stained with APC- or FITC-conjugated mAbs against mouse CD8 or CD4 and PE-conjugated anti-mouse IFN-γ (Biolegend). For intracellular IL-4 staining, cells were stimulated with ConA (3 μg/ml) for 2 days, followed by mouse IL-2 (10 ng/ml) and IL-4 (50 ng/ml) for 3 days. Cells were re-stimulated with immobilized CD3 (10 μg/ml) and soluble CD28 mAbs (2 μg/ml) in the presence of Golgiplug for 5 h. Cells were surface stained with anti-mouse CD4-APC and intracellularly stained with anti-mouse IL-4-FITC (eBiosciences).

In Vivo Cytotoxicity Assay.

B cells loaded with or without scFv-p3-4 were used as target cells for in vivo cytotoxicity assay. In brief, B cells were pulsed with scFv-p3-4 (10 μg/million cells) and then labeled with 2.5 μM CFSE (CFSE^{high}). Un-pulsed B cells were labeled with 0.25 μM CFSE (CFSE^{low}). The mixed B cells at a ratio of 1:1 were injected into mice immunized with different regimens. Mice were killed after 24 h of target cell transfer. Specific cytotoxicity was determined by detecting the differentially fluorescent-labeled target cell populations by flow cytometry. The percentage of cytotoxicity was determined as follows: (1−CFSE^{high}/CFSE^{low})×100%.

Flow Cytometry.

Splenocytes were incubated with anti-CD16/CD32 Fc receptor blocker for 10 min on ice and then washed and stained with indicated fluorochrome-conjugated mAbs. Cells were collected with a FACSCalibur flow cytometer (BD Immunocytometry Systems, San Jose, Calif.) and analyzed using FlowJo software (TreeStar, Ashland, Oreg.).

Mouse Immunization and Tumor Challenge.

Six- to 8-week-old Balb/c or C57Bl/6 mice were immunized i.v. with scFv, P3-4, or scFv-p3-4 at 50 μg/mouse/injection on days 0, 7 and 14. A group of mice immunized with PBS was used as control. On days 7 and 21, the sera were collected for her-2/neu Ab measurement. For tumor therapy in the prophylactic setting, mice were immunized 4 times and challenged by s.c. injection in the flank with 1×10⁵ D2F2/E2 (Balb/c) tumor cells. In the therapeutic setting, Balb/c or C57Bl/6 mice were first challenged with 1×10⁵ D2F2/E2 or 5×10⁵ EO771/E2 tumor cells. When palpable tumors formed, mice were treated with different regimens for 4 times at 1-week intervals. Tumor diameter was measured by calipers twice per week. Mice were killed when tumors reached 15 mm in diameter. In some experiments, survival was monitored up to 100 days beyond tumor implantation. In some experiments, mice were injected intraperitoneally with CD8 mAb (clone 2.43; 500 μg/mouse) or CD4 mAb (clone GK1.5; 250 μg/mouse) or isotype control mAb (250 μg/mouse) 3 days prior to immunization.

Statistical Analysis.

Unpaired T-test analysis was used to determine whether the differences between T and B cell-mediated immune responses induced by scFv-p3-4 versus scFv or P3-4 were significant. A two-way ANOVA and Kaplan-Meier survival analysis were used to determine significance for in vivo tumor therapy. P values less than 0.05 were considered significant.

### Example 1

### Generation of Anti-CD19 scFv Fusion Proteins that Specifically Bind to B Cells

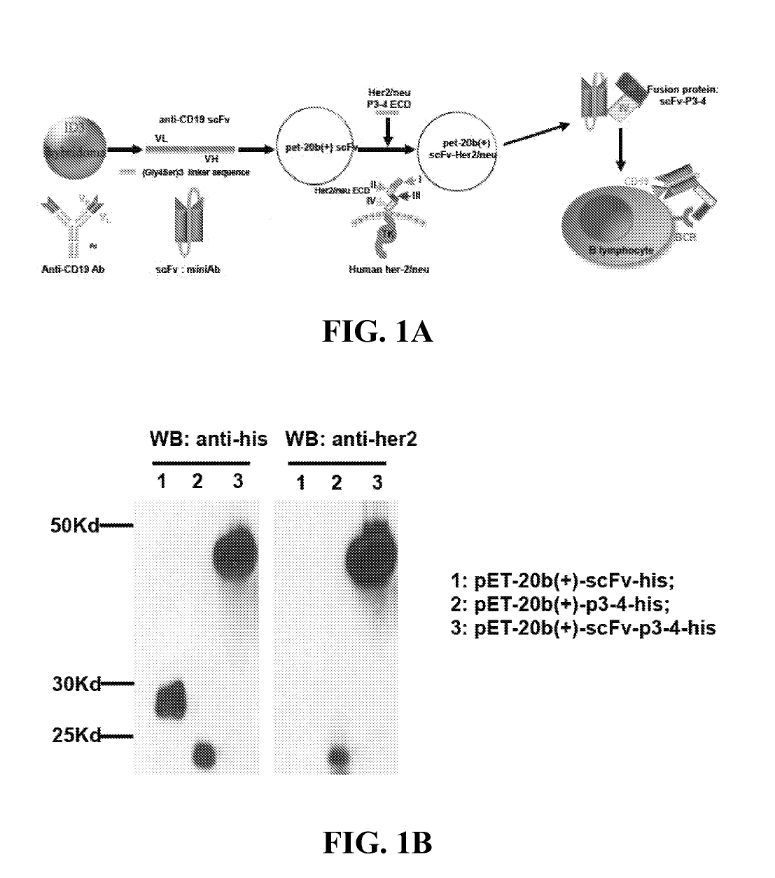

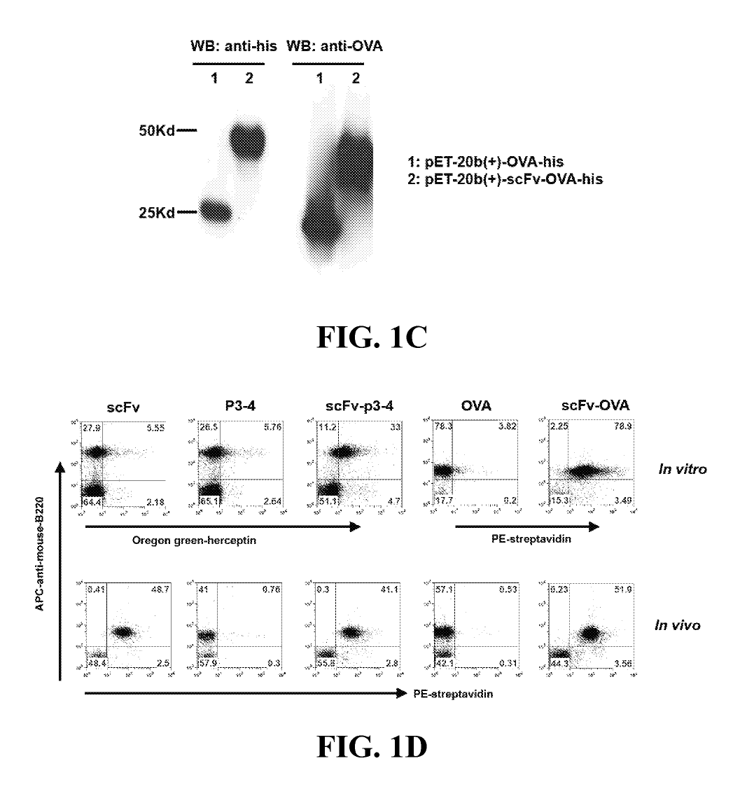

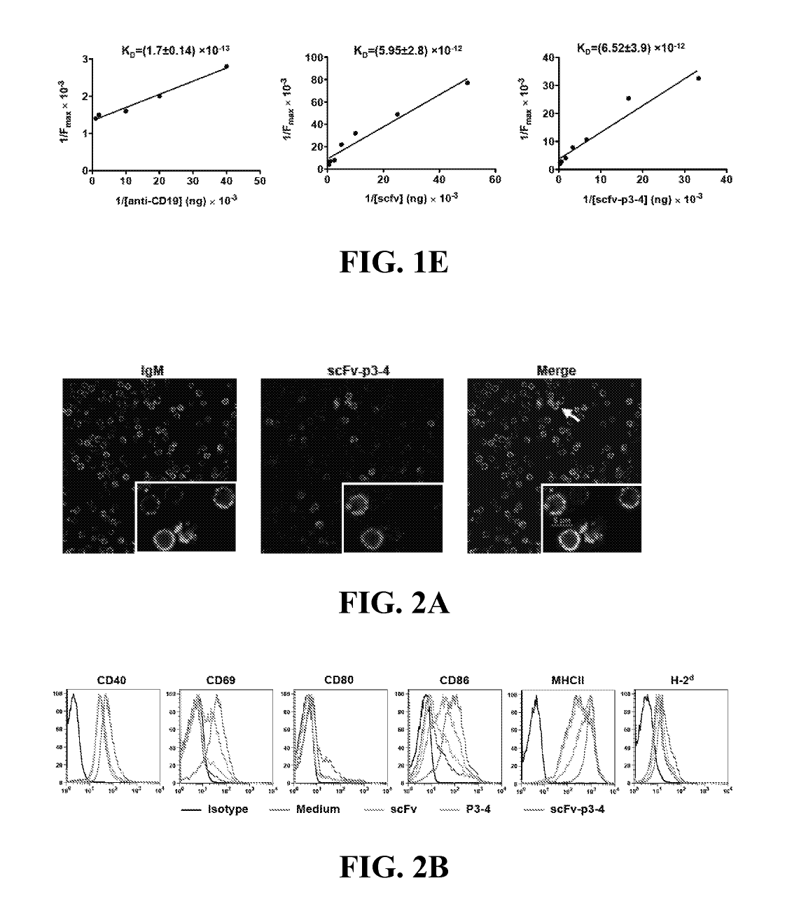

Previous studies have demonstrated that targeting of Ags via CD19 led to enhanced Ag-specific T cell responses and broke immune tolerance. In this regard, it was believed that if whole Abs were used, these could potentially stimulate the production of inflammatory cytokines resulting in serious adverse effects. Single chain Ab variable region fragments (scFv) are potentially useful as therapeutic reagents (see Norris (1996), Prechl (2002), and Ye (2002)) less likely to engender inflammatory responses. Single chain miniAbs are recombinant monovalent Abs lacking the constant part of both heavy and light chains. These molecules retain their Ag recognition ability and can be easily expressed in a prokaryotic system or mammalian cell lines. Anti-CD19 scFv miniAb were generated from 1D3 rat anti-mouse CD19 hybridoma cells. Candidate Ag genes such as her-2/neu ECD can be ligated with anti-CD19 scFv to make a fusion protein that specifically targets B cell CD19 and simultaneously engages the BCR (FIG. 1A). Tumor-associated Ag (TAA) her-2/neu ECD and surrogate Ag OVA were chosen to fuse with anti-CD19 scFv. The anti-CD19 scFv-her2/neu ECD cDNA constructs which contain cDNA that encodes the Herceptin-binding domain (from residues 475 to 652 amino acid, designated as P3-4) were generated. Similarly, truncated OVA cDNA (residues 241 to 386) was also ligated with scFv plasmids. Subsequently, these recombinant proteins were produced, purified, and characterized. As indicated in FIG. 1B, recombinant her-2/neu P3-4, anti-CD19 scFv-p3-4 (scFv-p3-4) fusion proteins were blotted positively with both His-Tag and her-2/neu Abs. The recombinant anti-CD19 scFv miniAb protein was blotted positively with His-Tag Ab but not with her-2/neu Ab nor OVA Ab. Similarly, recombinant proteins OVA and scFv-OVA reacted with His-Tag Ab and OVA Ab, respectively (FIG. 1C).

To verify that the scFv, scFv-p3-4 and scFv-OVA proteins retained the Ag-binding activity of the parental Ab, binding studies were conducted which measured specific binding to B cells in vitro and in vivo. For the in vitro binding assay, splenocytes were incubated with protein scFv, P3-4, scFv-p3-4, biotin-OVA, or biotin-scFv-OVA. For the in vivo binding assay, biotin-labeled-proteins were i.v. injected into mice. Peripheral blood was drawn at 10 min after injection. The successful targeting B cells was observed by the identification of double positive cells (FIG. 1D). These results indicate that anti-CD19 scFv miniAb with or without Ag tagged is capable of binding to B cells specifically. The protein Kd values were measured by Lineweaver-Burk analysis. The results revealed that scFv and scFv-p3-4 proteins retained high binding affinity to B cells (FIG. 1E).

### Example 2

### Fusion Protein scFv-p3-4 Activates B Cells to Produce Low Levels of Pro-Inflammatory Cytokines

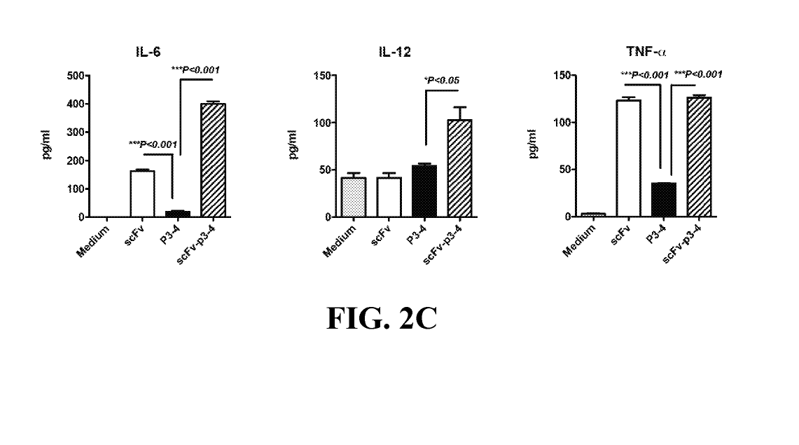

To determine whether Ag binding to B cells via anti-CD19 scFv was targeted to the BCR, B cells were incubated with anti-CD19 scFv-p3-4 fusion protein and stained with biotinylated anti-Her-2/neu followed by Alexa Fluor 594-streptavidin (red) and Alexa Fluor 488-anti-IgM (green). As shown in FIG. 2A, Ag linked to anti-CD19 scFv co-localized with surface IgM on B cells. To examine whether the fusion protein scFv-p3-4 could activate B cells, purified B cells were incubated with fusion proteins scFv, P3-4, scFv-p3-4. Expression of surface markers on B cells was assessed by flow cytometry. Fusion protein scFv-p3-4, but not P3-4, significantly up-regulated the expression levels of surface markers including CD40, CD69, CD80, CD86, MHC class II, and MHC class I molecule (FIG. 2B). Further cytokine measurement indicated that scFv-p3-4 stimulated low levels of pro-inflammatory cytokines including IL-6, IL-12P40, and TNF-α (FIG. 2C). scFv alone also stimulated moderate expression levels of CD69, CD86, and MHC class II but the levels were significantly lower than these stimulated by scFv-p3-4. In addition, scFv alone stimulated IL-6 and TNF-α production but not IL-12. These results provide evidence that Ag targeted to B cells via CD19 molecule can co-engage the BCR and stimulate full B cell activation.

### Example 3

### Targeting Surrogate Ag OVA to B Cells Via CD19 scFv Stimulates Augmented Ag-Specific CD4 and CD8 T-Cell Responses

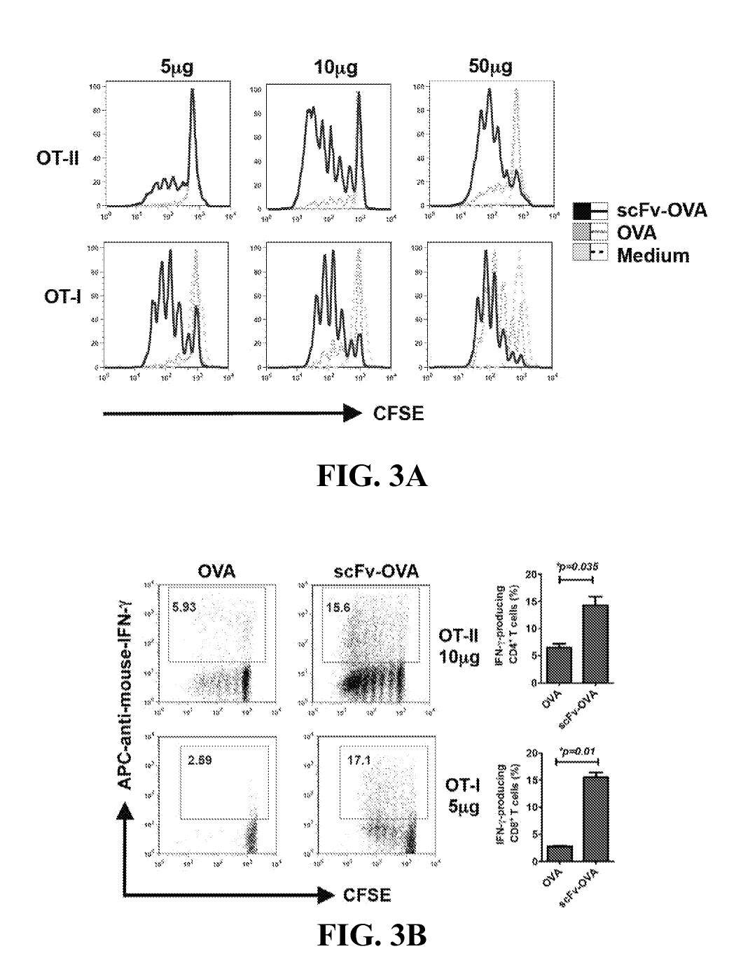

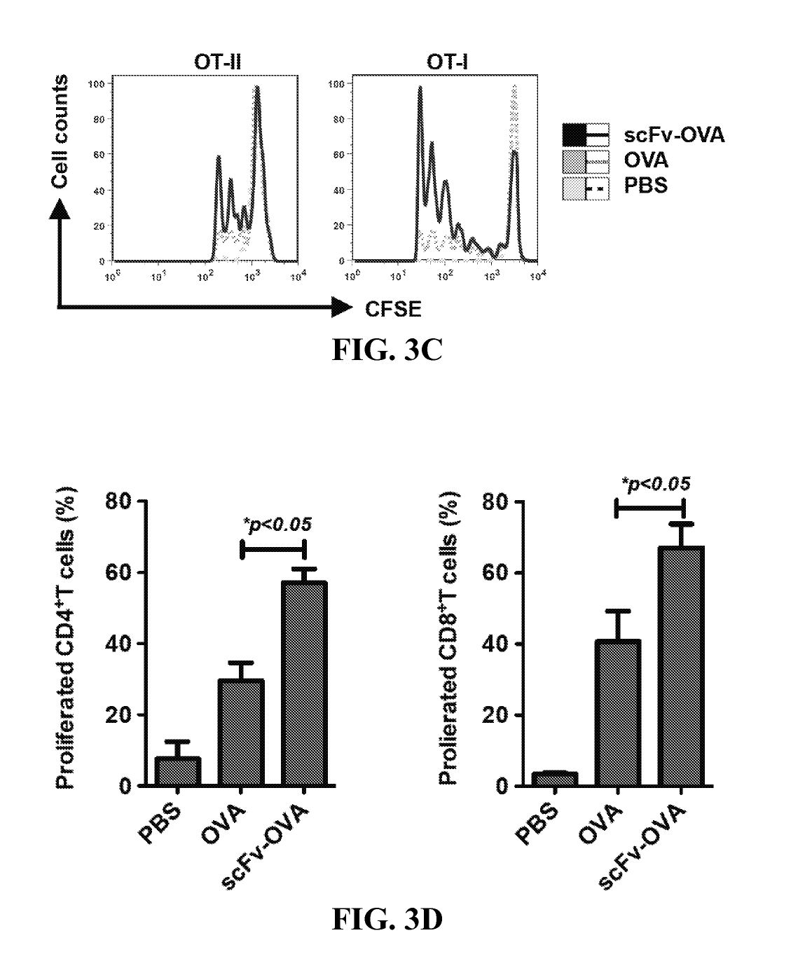

To determine whether targeting Ag to B cells increases CD4 and CD8 T-cell responses, the fusion protein scFv-OVA was generated for Ag presentation. For an in vitro Ag presentation assay, OT-II CD4⁺ T cells or OT-I CD8⁺T cells were used as readout of OVA Ag presentation. Indeed, CD4⁺ or CD8⁺ T cells underwent significantly more proliferation in response to scFv-OVA as compared to OVA stimulation (FIG. 3A). In addition, both CD4⁺ T and CD8⁺ T cells produced large amounts of IFN-γ upon scFv-OVA stimulation (FIG. 3B).

Additional studies were performed to examine whether this strategy would lead to enhanced T-cell proliferation in vivo. Mice were administered i.v. with 2×10⁶ CFSE-labeled naive OT-I or OT-II cells. The next day, mice were injected with soluble OVA or scFv-OVA. As an additional control, PBS was injected into another group of mice. As shown in FIG. 3C, both CD4⁺ and CD8⁺ T cells underwent at least 4 divisions within the first 3 days of exposure to scFv-OVA in vivo. In contrast, OT-I or OT-II T cells responded significantly less to the same amount of soluble OVA. These results provide evidence that scFv-OVA fusion protein induces potent T cell proliferation and differentiation both in vitro and in vivo.

### Example 4

### Herceptin-Like Anti-Tumor Abs are Elicited by Immunization with Fusion Protein scFv-p3-4

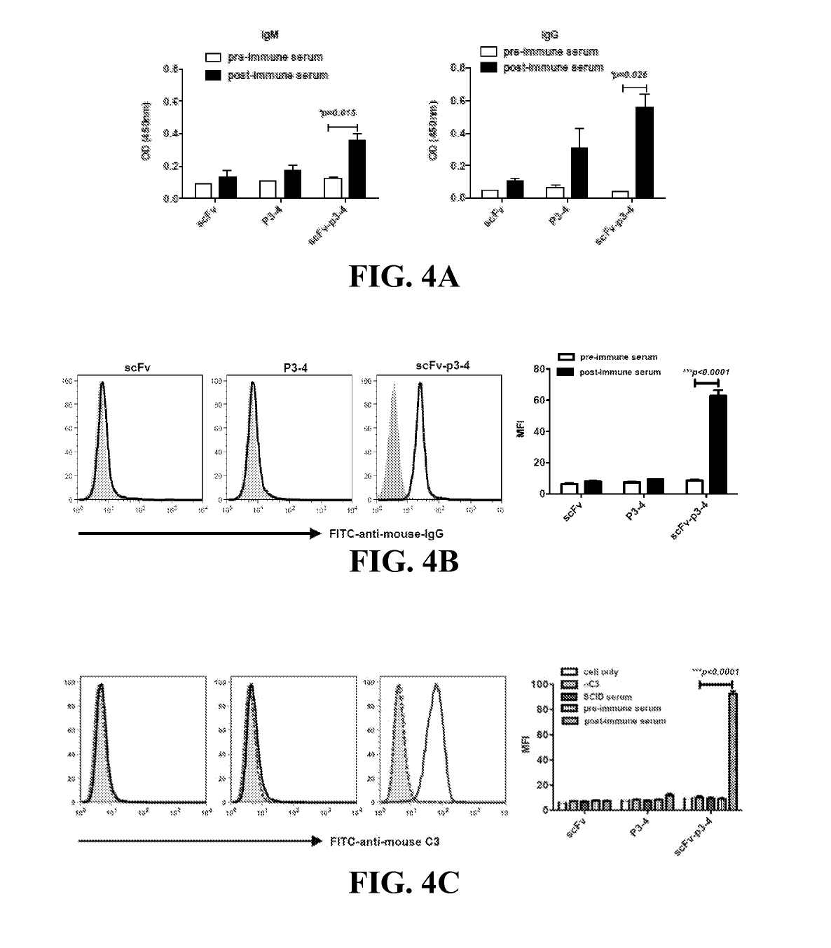

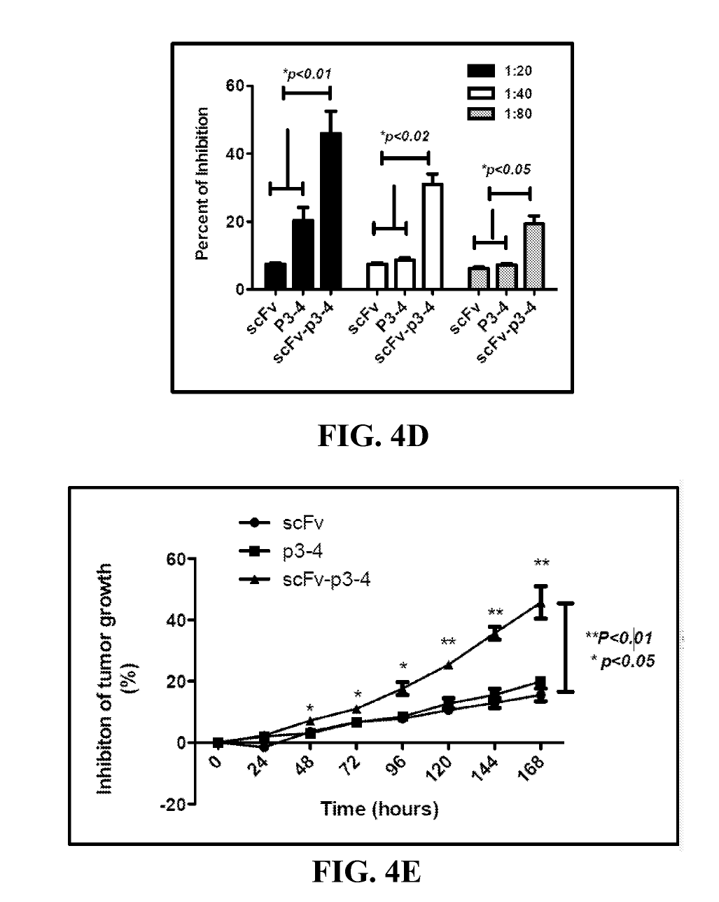

Further studies were conducted to test whether targeting TAA her-2/neu Ag to B cells could elicit anti-tumor Abs. As depicted in FIG. 4A, mice immunized with scFv-p3-4 secreted large amounts of her-2/neu Abs. P3-4 or scFv protein immunization did not elicit any appreciable level of her-2/neu Ab. The Ab specificity was further confirmed with her-2/neu-expressing human ovarian cell line SKOV-3 (FIG. 4B).

Next experiments were conducted to determine whether immune sera are capable of activating complement, one of the mechanisms for anti-tumor Ab-mediated tumor killing. Immune sera from scFv-p3-4 immunized mice showed potent complement activation (FIG. 4C). Since P3-4 contains the Herceptin-binding domain, whether Abs from mice immunized with scFv-p3-4 have Herceptin-like activity was examined. Competitive inhibition assay was performed in solid phase immunoassay with recombinant her-2/neu protein (P3-4) as the target Ag. The results revealed that the post-immune sera from mice immunized with scFv-p3-4, but not scFv or P3-4, were capable of competing with Herceptin binding (FIG. 4D). Approximately 50% inhibition was achieved when the immune sera were diluted at 1:20 (FIG. 4D).

The biological properties of Herceptin were first described for their ability to inhibit her-2/neu-positive human breast cancer cell growth in vitro (32) Inhibition of tumor cell growth in vitro is also an early and important indication of efficacy in vivo. As shown in FIG. 4E, sera from mice vaccinated with scFv-p3-4 significantly inhibited growth of human breast cancer cell SKBR-3. In contrast, sera from mice immunized with scFv or P3-4 showed minimal inhibition of tumor cell growth. Taken together, these results provide evidence that targeting her-2/neu p3-4 to B cells via CD19 scFv induces potent Ab response that activates complement and inhibits human breast cancer cell growth in vitro.

### Example 5

### Fusion Protein scFv-p3-4 Stimulates Enhanced CD4 Th2 Responses and her-2/neu-Specific CD8 T Cell Responses with Augmented Cytolytic Activity In Vivo

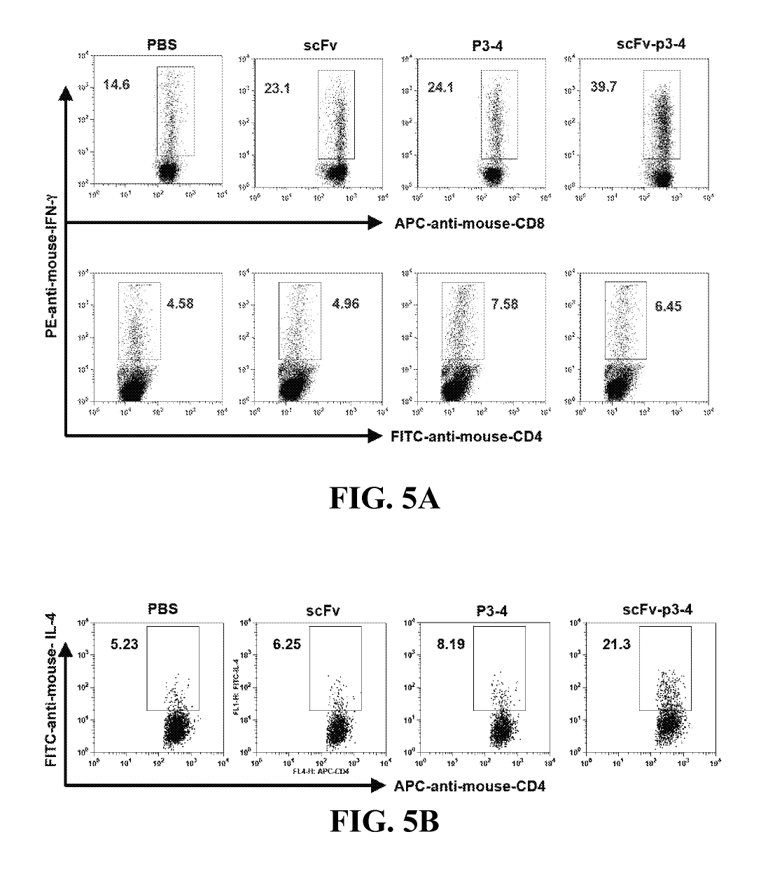

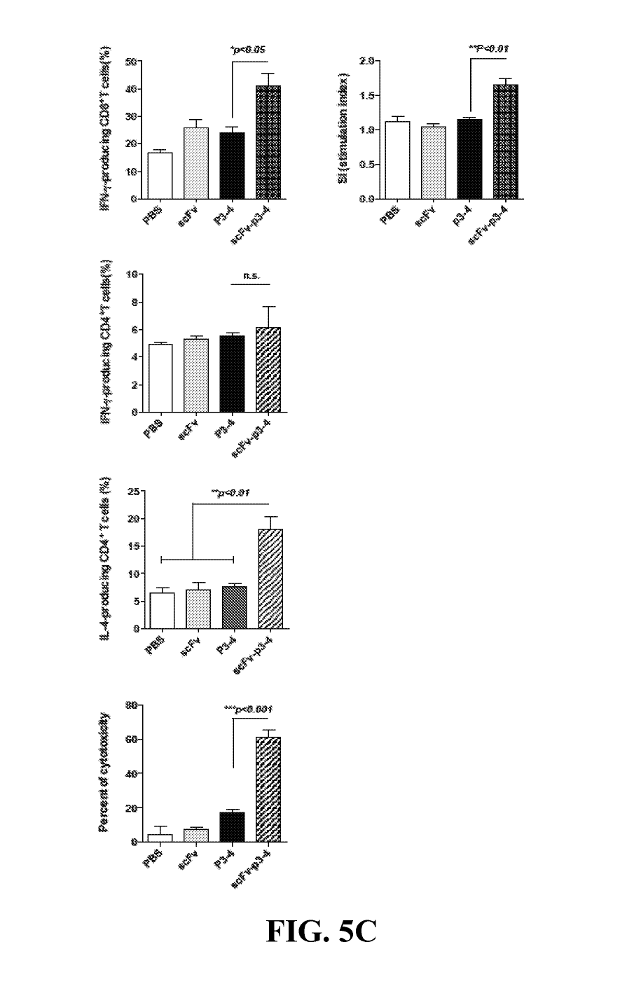

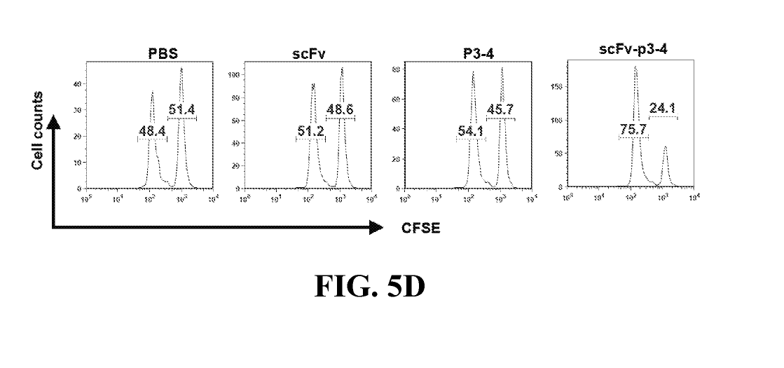

To determine whether anti-her-2/neu T-cell responses were elicited by this vaccination strategy, splenocytes from mice vaccinated with different fusion proteins were harvested and stimulated with scFv-p3-4 protein. It was found that IFN-γ-producing CD8⁺ T cells were significantly increased in scFv-p3-4 fusion protein immunized mice as compared to those from scFv-, P3-4-immunized or unimmunized mice (FIG. 5A). However, IFN-γ-producing CD4⁺ T cells were not significantly different among all groups of immunized mice (FIG. 5A). Since mice immunized with fusion protein scFv-p3-4 elicited potent humoral responses, we next examined IL-4 production from CD4⁺ T cells. As shown in FIG. 5B, CD4⁺ T cells from scFv-p3-4 immunized mice secreted significantly more IL-4 as compared to these from other fusion protein immunized mice. In addition, fusion protein scFv-p3-4, but not scFv or P3-4, prompted enhanced T cell proliferation as measured by ³H-thymidine incorporation (FIG. 5C). To determine the cytolytic activity against her-2/neu positive target cells, an in vivo cytotoxicity assay was performed. As shown in FIG. 5D, scFv-p3-4-immunized mice exhibited the highest cytolytic activity (mean=60%) versus less than 20% cytotoxicity in mice immunized with scFv or P3-4 alone (P<0.001).

### Example 6

### Vaccination with Fusion Protein scFv-p3-4 Induces Significant Anti-Tumor Effects

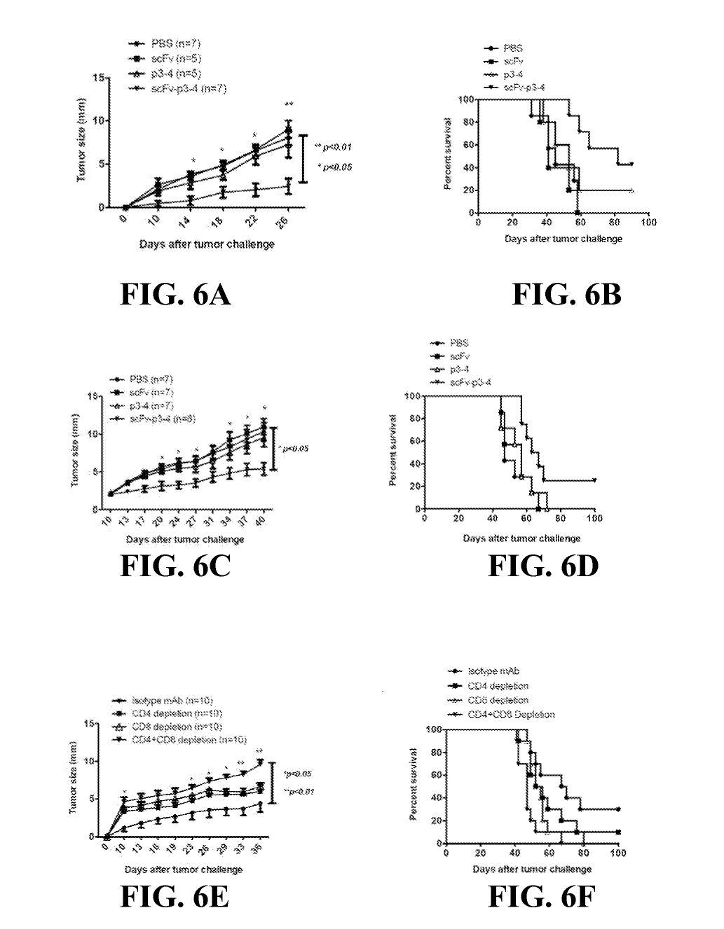

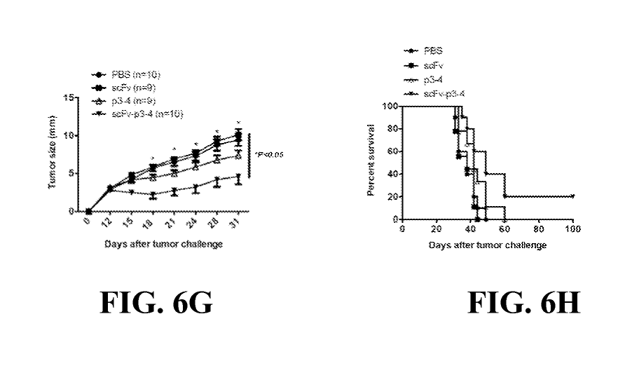

Since fusion protein scFv-p3-4 stimulated her-2/neu Ab response as well as enhanced CD8 T-cell responses in immunized mice, our next step was to determine whether anti-tumor immunity could be established by this vaccination approach. For the tumor prophylactic experiment, Balb/c mice were immunized i.v. with scFv, P3-4, or scFv-p3-4 on days 0, 7, 14 and 21. Mice immunized with different regimens were then challenged on day 28 by s.c. injection in the flank with 1×10⁵ syngeneic D2F2/E2 murine breast cancer cells that express human her-2/neu. As shown in FIG. 6A, mice immunized with scFv-p3-4 had a significantly delayed tumor progression compared with mice immunized with scFv, p3-4 or PBS control mice. In addition, these immunized mice achieved approximately 40% greater long-term, tumor-free survival (FIG. 6B). For the tumor therapeutic experiment, mice were first challenged with 1×10⁵ syngeneic her-2/neu-expressing D2F2/E2 tumor cells. Ten days after tumor inoculation, tumor-bearing mice were treated with fusion proteins scFv-p3-4, scFv, P3-4 or PBS at 1-week intervals.

Referring to FIG. 6C, the tumor-bearing mice treated with scFv-p3-4 had a significantly lower tumor burden compared with scFv, p3-4-treated mice or PBS control mice. In addition, these mice achieved approximately 25% long-term, tumor-free survival at day 100 (FIG. 6D). To gain insight into the cellular mechanisms of this vaccine, CD4⁺ and/or CD8⁺ T cells were depleted before mice were vaccinated. Her-2/neu Ab was not formed when CD4⁺ T cells were depleted (data not shown). As shown in FIG. 6E, mice depleted of both CD4⁺ and CD8⁺ cells completely lost scFv-p3-4-induced anti-tumor protection. Mice depleted of CD4⁺ or CD8⁺ cells showed increased tumor burden but not significantly different from isotype mAb-treated mice. These data provided evidence that both CD4⁺ and CD8⁺ are necessary for the scFv-p3-4-elicited anti-tumor immunity. To further confirm this therapeutic effect, C57Bl/6 mice implanted with human her-2/neu-expressing murine mammary carcinoma EO771 were treated with different regimens. Similar to the D2F2/E2 Babl/c model, the tumor-bearing mice treated with scFv-p3-4 had a significantly lower tumor burden compared with scFv, p3-4-treated mice or PBS control mice (FIG. 6G). In addition, these mice achieved approximately 20% long-term, tumor-free survival at day 100 (FIG. 6H).

### Example 7

### Targeting Different ECDs of her-2/neu to B Cells Elicits Potent Ab Responses

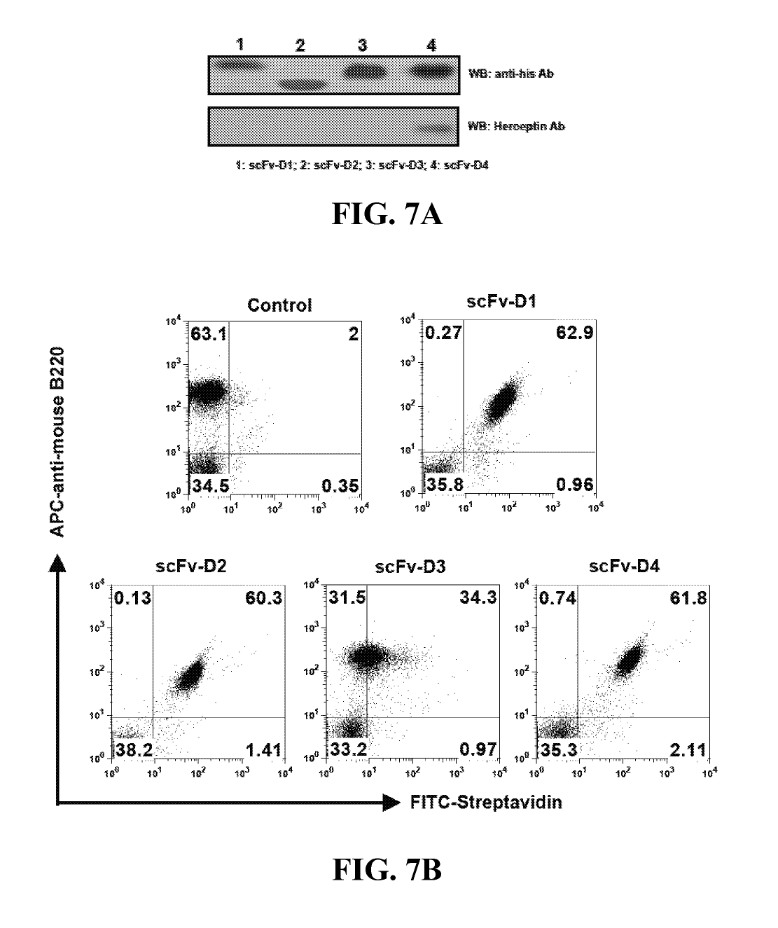

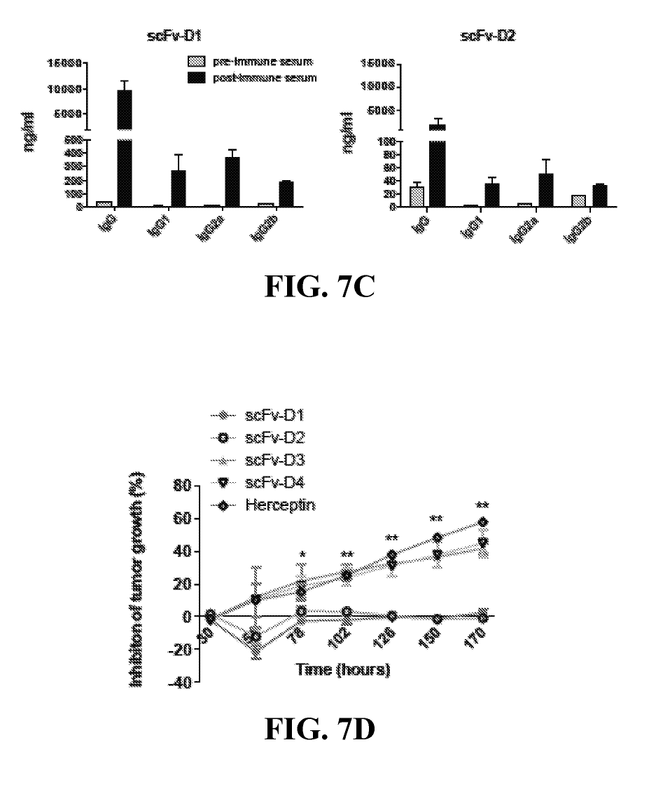

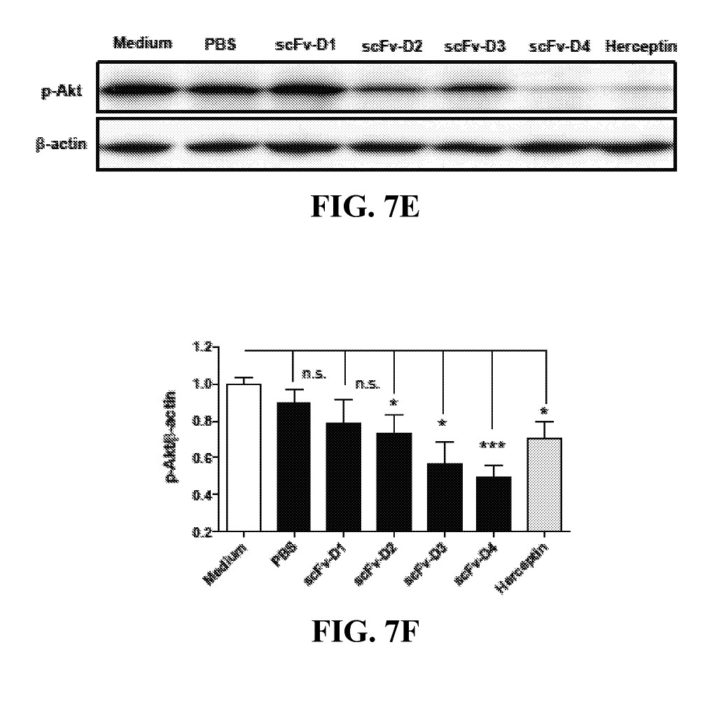

Since the her-2/neu ECD contains four different domains, experiments were conducted to investigate whether targeting different her-2/neu ECD domains to B cells could generate Abs specific to particular domains. Four different her-2/neu ECD domains were fused with CD19 scFv to generate fusion proteins. All fusion proteins blotted positively with His-Tag Ab but only scFv-D4 domain fusion protein blotted positively with Herceptin Ab (FIG. 7A). This is consistent with a previous report that the Herceptin-binding domain is located in the her-2/neu ECD D4 domain (see Cho (2003)). All fusion proteins bound to B cells with high affinity except scFv-D3 showed rather lower binding affinity to B cells as compared to other fusion proteins (FIG. 7B). Nevertheless, mice immunized with the four fusion proteins generated varying levels of IgG Ab levels with different isotypes (FIG. 7C). Interestingly, in vitro human breast cancer growth inhibition assays indicated that sera from scFv-D3 and scFv-D4 immune mice were as effective as Herceptin in causing growth inhibition (FIG. 7D). Despite a high titer of IgG Ab levels in mice immunized with scFv-D1, the serum did not show any inhibitory effect directly on human breast cancer cells (FIG. 7D). Furthermore, SKBR-3 human breast cancer cells constitutively express high levels of phospho-Akt. Herceptin treatment significantly inhibited p-Akt levels (FIG. 7E). Immune sera from scFv-D4 immunized mice showed similar inhibitory effects. Sera from scFv-D2 and scFv-D3 also showed a significant inhibitory effect on p-Akt levels. However, sera from scFv-D1 immunized mice did not show any effect on p-Akt level (FIGS. 7E and 7F). These data provided evidence that targeting different her-2/neu ECDs to B cells via CD19 is capable of generating Abs. However, these Abs could have differential biological effects.

### Discussion of Examples 1-7

One goal of the foregoing study was to generate a sustained anti-tumor Ab response as well as potent T cell responses. Although targeting Ags to DCs via lectins such as DEC205 and Clec9A has been shown to induce potent T cell responses (34-36), desirable B cell responses to any given Ag require direct contact between naïve B cells and intact Ag (3). Therefore, experiments were conducted that targeted Ags directly to B cells via a CD19 miniAb. That approach not only generated an augmented humoral response, but also potent T cell responses. The efficacy of this B cell-based vaccine was demonstrated in murine breast cancer models. CD4⁺ and CD8⁺ T cells may be both required for the vaccine to be most effective. In addition, this strategy can be used to generate Ab responses against any Ags of interest.

The uniqueness of the B cell-based vaccine approach is that Ags targeted to B cells elicit exaggerated Ag-specific Ab responses. DCs are conventionally considered as more potent APCs to induce both CD4⁺ and CD8⁺ T cell responses (Steinmann (2012)). They also indirectly promote B cell humoral responses. However, when Ags enter into DCs for the induction of Ab responses, Ags are processed and dominant epitopes are presented on the surface in the context of MHC class I or class II molecules. Generation of blocking or neutralizing Abs requires the presentation of intact Ag to B cells (Melchers (2012)). The inventors previously used intact CD19 mAb to target Ags specifically to B cells as reported in Yan (2005) and Ding (2008). However, intact mAb conjugates could potentially induce severe inflammatory responses. Indeed, a comparison study showed that whole CD19 mAb conjugates induced much more proinflammatory cytokines. Based on these results, a CD19 scFv miniAb was constructed and showed that these fusion proteins have high binding affinity to B cells although Kd values were lower than that with intact CD19 mAb. The co-engagement of CD19 and the BCR by fusion protein CD19 scFv-p3-4 activates B cells to upregulate the surface molecules CD40, CD80, CD86, MHC class I and II molecules that are critical for T cell activation and stimulation of low levels of cytokine production including IL-6, TNF-α, and IL-12. Although Ag-specific B cells are normally scarce, non-Ag specific B cells bound with fusion proteins via CD19 may serve as Ag-specific B cells for Ag presentation and T cell activation. However, it is unknown whether Ag internalization and further processing are required for B cell Ag presentation. In contrast, engagement of CD19 alone only induces moderate B cell activation and the BCR alone did not significantly stimulate B cell activation. Thus, it appears that full B cell activation requires co-engagement of CD19 and the BCR by scFv-p3-4 fusion protein. Previous studies have shown that production of IL-6 correlates with B cell vaccine efficacy via direct stimulation of CD8⁺ T cell proliferation (Vanden (2009)). IL-12 has also been shown to promote Th1 differentiation. Indeed, using the surrogate OVA Ag, the aforementioned studies demonstrate that fusion protein CD19 scFv-OVA elicited augmented CD4⁺ and CD8⁺ T cell proliferation as well as effector differentiation as revealed by more IFN-γ production.

This B cell-based vaccine strategy was extended to use TAA her-2/neu. Anti-human her-2/neu Ab Herceptin has been widely used for metastatic breast cancer patient care. It costs as much as US$70,000 per patient per year. In addition, a subset of breast cancer patients is refractory to Ab therapy despite high levels of her-2/neu expression on tumor cells. Furthermore, many patients who initially respond to Ab therapy ultimately develop resistance leading to disease progression. Previous studies demonstrated that her-2/neu-specific CD8 T cell responses could eradicate drug-refractory tumors.

The present disclosure demonstrates that targeting her-2/neu TAA to B cells via CD19 scFv miniAb elicited potent Ab responses. These Abs competitively inhibited Herceptin-binding ability. More importantly, these her-2/neu Abs are capable of activating complement and inhibiting her-2/neu⁺ human breast cancer cell line SKBR-3 growth. Additionally, her-2/neu-specific CD8 T cells were significantly enhanced in mice vaccinated with an scFv-p3-4 fusion protein. Although IFN-γ-producing CD4 T cells were not significant different among all groups, IL-4-producing CD4 T cells were significantly increased in scFv-p3-4-immunized mice which is consistent with potent Ab response elicited in these mice. This is in contrast to a recent report in which human her-2/neu protein was targeted to DCs via DEC-205 (44). In the presently-disclosed studies, DEC-her-2 vaccination with polyI:C as adjuvant induces potent T cell immunity. However, both DEC-her-2 or control her-2 protein induced similar levels of her-2/neu Ab response. It is unknown whether these Abs had tumor inhibitory activity.

The therapeutic efficacy of B cell-based vaccine was demonstrated using two murine breast cancer models. Human her-2/neu-expressing D2F2/E2 cells are refractory to Ab treatment but tumors can be controlled by CD8 T cells mediated by her-2 DNA vaccination. The present disclosure demonstrates that, a B cell-based vaccine has therapeutic efficacy in both prophylactic and therapeutic setting in terms of tumor progression. Tumor-free survival was also enhanced in these mice. Depletion of both CD4⁺ and CD8⁺ T cells completely abrogated the vaccine efficacy, indicating that both CD4⁺ and CD8⁺ T cells may both be required in some circumstances. CD4⁺ depletion also completely abolished the her-2/neu Ab response (data not shown), suggesting that although co-engagement of CD19 and BCR by fusion protein activates B cells, potent Ab production and isotype switching require CD4⁺ T cell help in some circumstance. It is worth noting that there was no adjuvant included in the current studies. Since B cells express multiple TLRs, addition of a TLR agonist such as CpG or polyI:C may significantly increase Ab and T cell responses. The therapeutic efficacy of this B cell vaccine was further tested in EO771 mammary carcinoma model on C57Bl/6 mice. Similarly, the tumor progression was significantly decreased in the tumor-bearing mice vaccinated with B cell vaccine.

The B cell-based vaccine strategy can be further extended to other areas including infectious disease, particularly for control of viral infection. Previous studies demonstrate that targeting HIV envelope glycoprotein trimers to B cells via a proliferation-inducing ligand (APRIL) induces potent Ab responses. Immunodominance is the concept that an antigenic determinant causes it to be responsible for the major immune response in a host. Immunodominance can occur in T and B cells. Ab immunodominance is reflected in the fact that the IgG response normally is specific for a single epitope. However, combating pathogens or cancer may require Ab and T cell responses against multiple epitopes to circumvent immune selection and escape. Targeting different her-2/neu ECD domains to B cells via CD19 generated Abs against each domain to varying levels. This approach may also offer a new way to generate mAbs. Interestingly, these Abs have different anti-tumor properties. Thus targeting selected, multiple epitopes to B cells may generate broader Ab and T cell responses that can clear pathogens or control tumor progression and recurrence. In summary, targeting Ags to B cells via CD19 miniAb generates both T and B cell responses. This vaccination approach provides a cost effective way to generate a sustained Herceptin-like Ab response as well as anti-tumor T cell responses.

Throughout this document, various references are mentioned. All such references are incorporated herein by reference, including the references set forth in the following list:

### REFERENCES

1. Kirkwood, J. M., L. H. Butterfield, A. A. Tarhini, H. Zarour, P. Kalinski, and S. Ferrone. 2012. Immunotherapy of cancer in 2012. CA: a cancer journal for clinicians.
2. Schlom, J. 2012. Therapeutic cancer vaccines: current status and moving forward. Journal of the National Cancer Institute 104: 599-613.
3. Kantoff, P. W., C. S. Higano, N. D. Shore, E. R. Berger, E. J. Small, D. F. Penson, C. H. Redfern, A. C. Ferrari, R. Dreicer, R. B. Sims, Y. Xu, M. W. Frohlich, and P. F. Schellhammer. 2010. Sipuleucel-T immunotherapy for castration-resistant prostate cancer. N Engl J Med 363: 411-422.
4. Krug, L. M., G. Ragupathi, C. Hood, C. George, F. Hong, R. Shen, L. Abrey, H. J. Jennings, M. G. Kris, and P. O. Livingston. 2012. Immunization with N-propionyl polysialic acid-KLH conjugate in patients with small cell lung cancer is safe and induces IgM antibodies reactive with SCLC cells and bactericidal against group B meningococci. Cancer Immunol Immunother 61: 9-18.
5. Tacken, P. J., I. J. de Vries, R. Torensma, and C. G. Figdor. 2007. Dendritic-cell immunotherapy: from ex vivo loading to in vivo targeting. Nat Rev Immunol 7: 790-802.
6. Dudziak, D., A. O. Kamphorst, G. F. Heidkamp, V. R. Buchholz, C. Trumpfheller, S. Yamazaki, C. Cheong, K. Liu, H. W. Lee, C. G. Park, R. M. Steinman, and M. C. Nussenzweig. 2007. Differential antigen processing by dendritic cell subsets in vivo. Science 315: 107-111.
7. Weiner, L. M., J. C. Murray, and C. W. Shuptrine. 2012. Antibody-based immunotherapy of cancer. Cell 148: 1081-1084.
8. Ferris, R. L., E. M. Jaffee, and S. Ferrone. 2010. Tumor antigen-targeted, monoclonal antibody-based immunotherapy: clinical response, cellular immunity, and immunoescape. J Clin Oncol 28: 4390-4399.
9. Constant, S. L. 1999. B lymphocytes as antigen-presenting cells for CD4+ T cell priming in vivo. J Immunol 162: 5695-5703.
10. Guo, K., J. Li, J. P. Tang, C. P. Tan, C. W. Hong, A. Q. Al-Aidaroos, L. Varghese, C. Huang, and Q. Zeng. 2011. Targeting intracellular oncoproteins with antibody therapy or vaccination. Science translational medicine 3: 99ra85.
11. Heit, A., K. M. Huster, F. Schmitz, M. Schiemann, D. H. Busch, and H. Wagner. 2004. CpG-DNA aided cross-priming by cross-presenting B cells. J Immunol 172: 1501-1507.
12. DiLillo, D. J., K. Yanaba, and T. F. Tedder. 2010. B cells are required for optimal CD4+ and CD8+ T cell tumor immunity: therapeutic B cell depletion enhances B16 melanoma growth in mice. J Immunol 184: 4006-4016.
13. de Visser, K. E., L. V. Korets, and L. M. Coussens. 2005. De novo carcinogenesis promoted by chronic inflammation is B lymphocyte dependent. Cancer cell 7: 411-423.
14. Fujimoto, M., Y. Fujimoto, J. C. Poe, P. J. Jansen, C. A. Lowell, A. L. DeFranco, and T. F. Tedder. 2000. CD19 regulates Src family protein tyrosine kinase activation in B lymphocytes through processive amplification. Immunity 13: 47-57.
15. Carter, R. H., and D. T. Fearon. 1992. CD19: lowering the threshold for antigen receptor stimulation of B lymphocytes. Science 256: 105-107.
16. Yan, J., M. J. Wolff, J. Unternaehrer, I. Mellman, and M. J. Mamula. 2005. Targeting antigen to CD19 on B cells efficiently activates T cells. Int Immunol 17: 869-877.
17. Ding, C., L. Wang, J. Marroquin, and J. Yan. 2008. Targeting of antigens to B cells augments antigen-specific T-cell responses and breaks immune tolerance to tumor-associated antigen MUC1. Blood 112: 2817-2825.
18. Baselga, J., and S. M. Swain. 2009. Novel anticancer targets: revisiting ERBB2 and discovering ERBB3. Nature reviews. Cancer 9: 463-475.
19. Slamon, D. J., B. Leyland-Jones, S. Shak, H. Fuchs, V. Paton, A. Bajamonde, T. Fleming, W. Eiermann, J. Wolter, M. Pegram, J. Baselga, and L. Norton. 2001. Use of chemotherapy plus a monoclonal antibody against HER2 for metastatic breast cancer that overexpresses HER2. N Engl J Med 344: 783-792.
20. Berns, K., H. M. Horlings, B. T. Hennessy, M. Madiredjo, E. M. Hijmans, K. Beelen, S. C. Linn, A. M. Gonzalez-Angulo, K. Stemke-Hale, M. Hauptmann, R. L. Beijersbergen, G. B. Mills, M. J. van de Vijver, and R. Bernards. 2007. A functional genetic approach identifies the PI3K pathway as a major determinant of trastuzumab resistance in breast cancer. Cancer cell 12: 395-402.
21. Shattuck, D. L., J. K. Miller, K. L. Carraway, 3rd, and C. Sweeney. 2008. Met receptor contributes to trastuzumab resistance of Her2-overexpressing breast cancer cells. Cancer Res 68: 1471-1477.
22. Zhang, S., W. C. Huang, P. Li, H. Guo, S. B. Poh, S. W. Brady, Y. Xiong, L. M. Tseng, S. H. Li, Z. Ding, A. A. Sahin, F. J. Esteva, G. N. Hortobagyi, and D. Yu. 2011. Combating trastuzumab resistance by targeting SRC, a common node downstream of multiple resistance pathways. Nat Med 17: 461-469.
23. Whittington, P. J., M. P. Piechocki, H. H. Heng, J. B. Jacob, R. F. Jones, J. B. Back, and W. Z. Wei. 2008. DNA vaccination controls Her-2+ tumors that are refractory to targeted therapies. Cancer Res 68: 7502-7511.
24. Fleck, L. M. 2006. The costs of caring: Who pays? Who profits? Who panders? The Hastings Center report 36: 13-17.
25. Radkevich-Brown, O., J. Jacob, M. Kershaw, and W. Z. Wei. 2009. Genetic regulation of the response to Her-2 DNA vaccination in human Her-2 transgenic mice. Cancer Res 69: 212-218.
26. Benedict, C. A., A. J. MacKrell, and W. F. Anderson. 1997. Determination of the binding affinity of an anti-CD34 single-chain antibody using a novel, flow cytometry based assay. Journal of immunological methods 201: 223-231.
27. Li, B., D. J. Allendorf, R. Hansen, J. Marroquin, C. Ding, D. E. Cramer, and J. Yan. 2006. Yeast beta-glucan amplifies phagocyte killing of iC3b-opsonized tumor cells via complement receptor 3-Syk-phosphatidylinositol 3-kinase pathway. J Immunol 177: 1661-1669.
28. Solly, K., X. Wang, X. Xu, B. Strulovici, and W. Zheng. 2004. Application of real-time cell electronic sensing (RT-CES) technology to cell-based assays. Assay Drug Dev Technol 2: 363-372.
29. L. K., N. A. Norris, H. Marquardt, T. T. Tsu, M. S. Hayden, M. G. Neubauer, D. E. Yelton, R. S. Mittler, and J. A. Ledbetter. 1996. Rapid and reliable cloning of antibody variable regions and generation of recombinant single chain antibody fragments. Tissue Antigens 47: 1-20.
30. Prechl, J., D. C. Baiu, A. Horvath, and A. Erdei. 2002. Modeling the presentation of C3d-coated antigen by B lymphocytes: enhancement by CR1/2-BCR co-ligation is selective for the co-ligating antigen. Int Immunol 14: 241-247.
31. Ye, Z., I. Hellstrom, M. Hayden-Ledbetter, A. Dahlin, J. A. Ledbetter, and K. E. Hellstrom. 2002. Gene therapy for cancer using single-chain Fv fragments specific for 4-1BB. Nat Med 8: 343-348.
32. Baselga, J., L. Norton, J. Albanell, Y. M. Kim, and J. Mendelsohn. 1998. Recombinant humanized anti-HER2 antibody (Herceptin) enhances the antitumor activity of paclitaxel and doxorubicin against HER2/neu overexpressing human breast cancer xenografts. Cancer Res 58: 2825-2831.
33. Cho, H. S., K. Mason, K. X. Ramyar, A. M. Stanley, S. B. Gabelli, D. W. Denney, Jr., and D. J. Leahy. 2003. Structure of the extracellular region of HER2 alone and in complex with the Herceptin Fab. Nature 421: 756-760.
34. Charalambous, A., M. Oks, G. Nchinda, S. Yamazaki, and R. M. Steinman. 2006. Dendritic cell targeting of survivin protein in a xenogeneic form elicits strong CD4+ T cell immunity to mouse survivin. J Immunol 177: 8410-8421.
35. Idoyaga, J., A. Lubkin, C. Fiorese, M. H. Lahoud, I. Caminschi, Y. Huang, A. Rodriguez, B. E. Clausen, C. G. Park, C. Trumpfheller, and R. M. Steinman. 2011. Comparable T helper 1 (Th1) and CD8 T-cell immunity by targeting HIV gag p24 to CD8 dendritic cells within antibodies to Langerin, DEC205, and Clec9A. Proc Natl Acad Sci USA 108: 2384-2389.
36. Caminschi, I., A. I. Proietto, F. Ahmet, S. Kitsoulis, J. Shin Teh, J. C. Lo, A. Rizzitelli, L. Wu, D. Vremec, S. L. van Dommelen, I. K. Campbell, E. Maraskovsky, H. Braley, G. M. Davey, P. Mottram, N. van de Velde, K. Jensen, A. M. Lew, M. D. Wright, W. R. Heath, K. Shortman, and M. H. Lahoud. 2008. The dendritic cell subtype-restricted C-type lectin Clec9A is a target for vaccine enhancement. Blood 112: 3264-3273.
37. Melchers, M., I. Bontjer, T. Tong, N. P. Chung, P. J. Klasse, D. Eggink, D. C. Montefiori, M. Gentile, A. Cerutti, W. C. Olson, B. Berkhout, J. M. Binley, J. P. Moore, and R. W. Sanders. 2012. Targeting HIV-1 envelope glycoprotein trimers to B cells by using APRIL improves antibody responses. J Virol 86: 2488-2500.
38. Steinman, R. M. 2012. Decisions about dendritic cells: past, present, and future Annu Rev Immunol 30: 1-22.
39. Vanden Bush, T. J., C. M. Buchta, J. Claudio, and G. A. Bishop. 2009. Cutting Edge: Importance of IL-6 and cooperation between innate and adaptive immune receptors in cellular vaccination with B lymphocytes. J Immunol 183: 4833-4837.
40. Romond, E. H., E. A. Perez, J. Bryant, V. J. Suman, C. E. Geyer, Jr., N. E. Davidson, E. Tan-Chiu, S. Martino, S. Paik, P. A. Kaufman, S. M. Swain, T. M. Pisansky, L. Fehrenbacher, L. A. Kutteh, V. G. Vogel, D. W. Visscher, G. Yothers, R. B. Jenkins, A. M. Brown, S. R. Dakhil, E. P. Mamounas, W. L. Lingle, P. M. Klein, J. N. Ingle, and N. Wolmark. 2005. Trastuzumab plus adjuvant chemotherapy for operable HER2-positive breast cancer. N Engl J Med 353: 1673-1684.
41. Nahta, R. 2012. Pharmacological strategies to overcome HER2 cross-talk and Trastuzumab resistance. Current medicinal chemistry 19: 1065-1075.
42. Nahta, R., and R. M. O'Regan. 2012. Therapeutic implications of estrogen receptor signaling in HER2-positive breast cancers. Breast cancer research and treatment.
43. Whittington, P. J., O. Radkevich-Brown, J. B. Jacob, R. F. Jones, A. M. Weise, and W. Z. Wei. 2009. Her-2 DNA versus cell vaccine: immunogenicity and anti-tumor activity. Cancer Immunol Immunother 58: 759-767.
44. Wang, B., N. Zaidi, L. Z. He, L. Zhang, J. M. Kuroiwa, T. Keler, and R. M. Steinman. 2012. Targeting of the non-mutated tumor antigen HER2/neu to mature dendritic cells induces an integrated immune response that protects against breast cancer in mice. Breast Cancer Res 14: R39.
45. Rawlings, D. J., M. A. Schwartz, S. W. Jackson, and A. Meyer-Bahlburg. 2012. Integration of B cell responses through Toll-like receptors and antigen receptors. Nat Rev Immunol 12: 282-294.
46. Yewdell, J. W., and J. R. Bennink 1999. Immunodominance in major histocompatibility complex class I-restricted T lymphocyte responses. Annu Rev Immunol 17: 51-88.

It will be understood that various details of the presently-disclosed subject matter can be changed without departing from the scope of the subject matter disclosed herein. Furthermore, the foregoing description is for the purpose of illustration only, and not for the purpose of limitation.

What is claimed is:

1. A method of eliciting an immune response, comprising contacting a B cell with an effective amount of a composition comprising an anti-CD19 single chain variable fragment (scFv) conjugated to a tumor-associated antigen to thereby elicit the immune response.

2. The method of claim 1, wherein the tumor associated antigen comprises a Her-2/neu protein.

3. The method of claim 2, wherein the Her-2/neu protein comprises a Her-2/neu extracellular domain.

4. The method of claim 1, wherein the immune response comprises B cell antibody generation, T cell activation, T cell differentiation, or combinations thereof.

5. The method of claim 1, wherein the immune response comprises an increase in an amount of one or more inflammatory cytokines.

6. A method of treating a cancer, comprising administering to a subject in need thereof an effective amount of a composition comprising an anti-CD19 single chain variable fragment (scFv) conjugated to a tumor-associated antigen.

7. The method of claim 6, wherein the tumor associated antigen comprises a Her-2/neu protein.

8. The method of claim 7, wherein the Her-2/neu protein comprises a Her-2/neu extracellular domain.

9. The method of claim 6, wherein the administering to a subject comprises repeated administered doses of the composition.

10. The method of claim 6, wherein the administering the composition elicits an immune response.

11. A method of limiting the occurrence of cancer in a subject, the method comprising administering a composition comprising an anti-CD19 single chain variable fragment (scFv) conjugated to a tumor-associated antigen, to a subject in need thereof, in an amount that generates an immune response to thereby limit the occurrence of cancer in the subject.

12. The method of claim 11, wherein the administering the composition comprises repeated administrative doses of the composition, which results in the immune response.

13. The method of claim 11, wherein the tumor associated antigen comprises a Her-2/neu protein.

14. The method of claim 11, wherein the cancer is a breast cancer.


SEQUENCE LISTING (14 sequences)

SEQ ID NO: 1 - DNA, 40 bp, Artificial Sequence
  PRIMER SEQUENCE
gaagatctccaccatggacattcagctgacccagtctcca

SEQ ID NO: 2 - DNA, 39 bp, Artificial Sequence
  primer sequence
agagccacctccgccccgtttcagttccagcttggtgcc

SEQ ID NO: 3 - DNA, 37 bp, Artificial Sequence
  primer sequence
ggcggaggtggctctargtsmarctgvwgsartcwgg

SEQ ID NO: 4 - DNA, 45 bp, Artificial Sequence
  primer sequence
actagtcgactcatgaggacacggtgaccatggttcctgggcccc

SEQ ID NO: 5 - DNA, 29 bp, Artificial Sequence
  primer sequence
aatgtcgacatggagctggcggccttgtg

SEQ ID NO: 6 - DNA, 30 bp, Artificial Sequence
  primer sequence
aatctcgagacaggggtggcaggcccgaga

SEQ ID NO: 7 - DNA, 31 bp, Artificial Sequence
  primer sequence
aatgtcgactctccgatgtgtaagggctccc

SEQ ID NO: 8 - DNA, 30 bp, Artificial Sequence
  primer sequence
aatctcgagttggttgtgcagggggcagac

SEQ ID NO: 9 - DNA, 30 bp, Artificial Sequence
  primer sequence
aatgtcgacgaggtgacagcagaggatgga

SEQ ID NO: 10 - DNA, 30 bp, Artificial Sequence
  primer sequence
aatctcgaggttccgaaagagctggtccca

SEQ ID NO: 11 - DNA, 30 bp, Artificial Sequence
  primer sequence
aatgtcgacccgcaccaagctctgctccac

SEQ ID NO: 12 - DNA, 29 bp, Artificial Sequence
  primer sequence
aatctcgagcgtcagagggctggctctct

SEQ ID NO: 13 - DNA, 27 bp, Artificial Sequence
  primer sequence
aggtcgacagcatgttggtgctgttgc

SEQ ID NO: 14 - DNA, 27 bp, Artificial Sequence
  primer sequence
gactcgagaggggaaacacatctgcca